(19) United States
(12) Patent Application Publication
(10) Pub. No.: US 2023/0301972 A1
(43) Pub. Date: Sep. 28, 2023

(54) ANTI-FIBROTIC COMPOSITION AND RELATED METHODS

(71) Applicant: Fred Hutchinson Cancer Center, Seattle, WA (US)

(72) Inventors: Marina Chan, Seattle, WA (US); Taranjit Gujral, Seattle, WA (US)

(73) Assignee: Fred Hutchinson Cancer Center, Seattle, WA (US)

(21) Appl. No.: 17/999,240

(22) PCT Filed: May 19, 2021
(86) PCT No.: PCT/US2021/033080

§ 371 Date: Nov. 18, 2022

Related U.S. Application Data
(60) Provisional application No. 63/027,645, filed on May 20, 2020.

(51) Int. Cl. A61K 31/427; A61P 11/00; A61P 1/16; A61K 31/496; A61K 31/4418

(52) U.S. Cl. CPC A61K 31/427; A61P 11/00; A61P 1/16; A61K 31/496; A61K 31/4418

(57) ABSTRACT

The present disclosure is based on the discovery that a clinical grade compound of Formula (I) potently blocks TGF-β activity and significantly reverse the activated phenotypes of myofibroblast in vitro. The compound of Formula (I) can be used in the treatment of fibrotic conditions in general, such as non-alcoholic steatohepatitis (NASH), cirrhosis, HBV infection, any liver disease, pulmonary fibrosis, interstitial lung disease, idiopathic pulmonary fibrosis (IPF), renal fibrosis, cardiac fibrosis, or any combination thereof.

### CROSS-REFERENCE(S) TO RELATED APPLICATION(S)

This application claims the benefit of U.S. Patent Application No. 63/027,645, filed May 20, 2020, the disclosure of which is hereby incorporated by reference in its entirety.

### STATEMENT OF GOVERNMENT LICENSE RIGHTS

This invention was made with Government support under CA201229 awarded by the National Institutes of Health. The Government has certain rights in the invention.

### BACKGROUND

Organ fibrosis is a major cause of morbidity and mortality. Fibrosis is a common pathway for progression of many debilitating diseases associated with loss of organ function, including Non-Alcoholic SteatoHepatitis (“NASH” in the liver) and Idiopathic Pulmonary Fibrosis (“IPF” in the lung), which can require organ transplantation to delay mortality and/or reduce morbidity. Accordingly, such diseases represent significant unmet medical needs.

### Liver Fibrosis

An exemplary risk factor for liver fibrosis is infection with hepatitis B virus. The latest clinical practice guidelines published by European Association for the Study of Liver (EASL) point out that about one-third of the world's population has been or is infected with hepatitis B virus (HBV). Among those who end up with chronic infection, between 15% and 40% will develop progressive liver disease in terms of fibrosis over time. Annually about 3% patients with chronic infection develop cirrhosis. Characterized by diffuse hepatic fibrosis and nodule formation, hepatitis B cirrhosis mainly contains compensated and decompensated stage. Compensated cirrhosis can further develop into decompensated stage and hepatocellular carcinoma with serious complications and high mortality. The 5-year survival rates of patients with liver cancer of all stages combined, compensated and decompensated cirrhosis are 18%, 84% and 14%, respectively. Overall, HBV patients are at particularly high risk for developing liver fibrosis.

Liver fibrosis is characterized by the excessive accumulation of extracellular matrix (ECM) due to chronic liver injury of virtually any etiology including chronic HBV infection, alcohol or drug abuse, nonalcoholic steatohepatitis and autoimmune imbalances. Though the tissue fibrotic process is complex and dynamic, the activation of myofibroblasts is a major contributor to the net production of ECM and induction of a pro-fibrotic microenvironment. The accumulation of ECM proteins distorts the hepatic architecture by forming fibrous scars, and the subsequent development of nodules of regenerating hepatocytes defines advanced fibrosis and cirrhosis. Historically, fibrosis was thought to be an intractable and irreversible process, though research in the past few decades has redefined it as a model of the wound-healing response to chronic liver injury, for which there are potentially new opportunities for reversibility. Liver injuries elicit a complex and dynamic (both pro-regenerative/anti-fibrotic and pro-fibrotic) response of resident hepatic cells, including hepatocytes, Kupffer cells, sinusoidal endothelial cells and hepatic stellate cells, and infiltrating immune cells including natural killer cells, natural killer T cells, monocytes, and macrophages. Together, these resident hepatic cells maintain the liver regenerative capacity in the absence of persistent injury. Eventually the remarkable regenerative and healing capacity of the liver succumbs to the continued injurious exposure to many of these secreted signals along with the accumulation of ECM.

During liver fibrosis, the activation of hepatic stellate cells (HSCs), which are hepatic resident mesenchymal cells, contributes to the net production of ECM and induction of a pro-fibrotic microenvironment. Under physiological conditions, HSCs reside in the perisinusoidal space in between the hepatocytes and sinusoidal endothelial cells and maintain a quiescent state (qHSCs). qHSCs store retinoid lipid droplets and have extensive dendrite-like processes along the sinusoid. Following injury in vivo or in vitro culturing, quiescent HSCs transdifferentiate into myofibroblasts. In this process, they lose their retinoid droplets, enlarge and flatten, express αSMA and vimentin, and become more proliferative, migratory, and proinflammatory. They synthesize large quantities of extracellular matrix molecules (e.g., type I and III collagens) and other ECM remodeling proteins (e.g., MMPs and TIMPs).

### Idiopathic Pulmonary Fibrosis

Idiopathic pulmonary fibrosis (IPF) is a chronic, progressive, and fibrotic interstitial lung disease of unknown origin. Patients with IPF present progressive difficulty in breathing, pulmonary hypertension, and respiratory failure. In the United States, 128,000 people are affected with IPF and 48,000 new cases are diagnosed each year. IPF patients have poor prognosis with a median survival rate of just under three years, representing a significant unmet medical need.

IPF is characterized by alveolar epithelial cell damage, subsequent release of pro-inflammatory and pro-fibrotic cytokines and other mediators, and excessive abnormal deposition of extracellular matrix (ECM) proteins. Though the pathogenic mechanisms underlying IPF are not yet fully understood, it is well established that a major culprit is TGF-β driven chemotaxis and proliferation of lung fibroblasts, differentiation of fibroblasts into myofibroblasts as well as production of profibrotic molecules which promote ECM deposition. Myofibroblasts are the key cell types that contributes to the net production of ECM and induction of a pro-fibrotic microenvironment. They are a specialized contractile type of cells that predominate in areas of active fibrosis, namely, fibroblastic foci. Lung fibroblasts can migrate in the site of injury attracted by mediators such as platelet-derived growth factor (PDGF) and transforming growth factor-β (TGF-β) produced by injured alveolar epithelial cells and differentiate into myofibroblasts. These activated lung fibroblasts/myofibroblasts upregulate expression of various ECM proteins including collagens, integrins, and fibronectin (FN1) that regulate cell adhesion, migration and proliferation. TGF-β is considered to be the most potent fibrogenic and proliferative signal to stimulate myofibroblast activation. Further, several other growth factors including FGF basic, VEGF, CTGF and PDGF have all been implicated in myofibroblast activation and function. Further, recent study has shown that several fibrosis-driving cytokines like TGFb and CTGF feed into a common pathway, IL-11, making it an “attractive nexus of profibrotic factors.

Currently, there are two approved drugs for treating IPF, both targeting fibroblasts, by inhibiting multi-kinases (nintedanib) or TGF-β pathway (pirfenidone). However, both of these drugs only slow disease progression in a portion of patients with significant and sometimes intolerable adverse effects. Accordingly, a need remains for safe and effective interventions to prevent, treat, and/or reverse the progression of fibrosis.

### Extracellular Signals

As illustrated above, a panoply of extracellular signals has been found to affect myofibroblasts function and phenotype during organ injury. TGF-β is widely considered to be the most potent fibrogenic and proliferative signal to stimulate myofibroblast activation both in lung and liver. Binding of TGF-β to its receptors and subsequent activation of its downstream component SMAD2/3 promotes transcription of type I and type III collagens. Other signals such as growth factor signals including PDGF, FGF, VEGF and CTGF, as well as negative regulators of hepatic myofibroblasts (e.g., nuclear receptors FXR, PPARs) have all been demonstrated in vitro and/or in vivo to dampen the activated phenotypes of myofibroblasts.

However, despite the advances in characterizing the molecular signals underlying myofibroblast regulation, there remains a paucity of approved anti-fibrosis therapeutics. Accordingly, a need remains for safe and effective interventions to prevent, treat, and/or reverse the progression of organ fibrosis. The present disclosure addresses this and related needs and presents further advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features a method of inhibiting signaling mediated by TGF-β, FGF, VEGF, PDGF, and/or a Src family kinase in a cell, including contacting the cell with a therapeutically effective amount of a compound of formula (I)

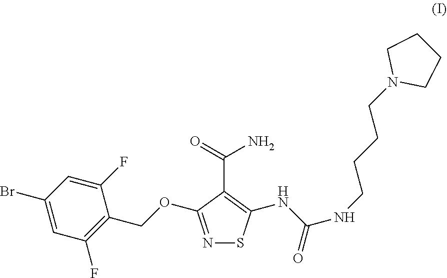 [SMILES: NC(=O)c1c(OCc2c(F)cc(Br)cc2F)nsc1NC(=O)NCCCCN1CCCC1]

or a pharmaceutically acceptable salt, prodrug, or solvate thereof.

In another aspect, the present disclosure features a method of inhibiting activity of a myofibroblast or fibroblast, including contacting the myofibroblast or fibroblast with a therapeutically effective amount of a compound of formula (I)

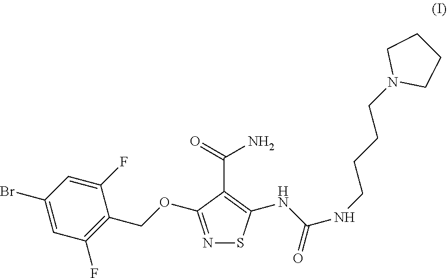 [SMILES: NC(=O)c1c(OCc2c(F)cc(Br)cc2F)nsc1NC(=O)NCCCCN1CCCC1]

or a pharmaceutically acceptable salt, prodrug, or solvate thereof.

In yet another aspect, the present disclosure features a method of increasing hepatocyte regeneration in a subject in need thereof, including administering to the subject a therapeutically effective amount of a pharmaceutical composition including a compound of Formula (I)

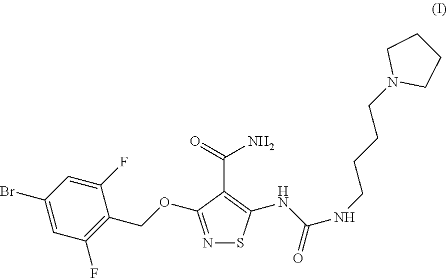 [SMILES: NC(=O)c1c(OCc2c(F)cc(Br)cc2F)nsc1NC(=O)NCCCCN1CCCC1]

or a pharmaceutically acceptable salt, prodrug, or solvate thereof, and a pharmaceutically acceptable carrier.

In yet a further aspect, the present disclosure features a method of treating a fibrosis condition in a subject in need thereof, comprising administering to subject a therapeutically effective amount of a first pharmaceutical composition including:
a compound of formula (I)

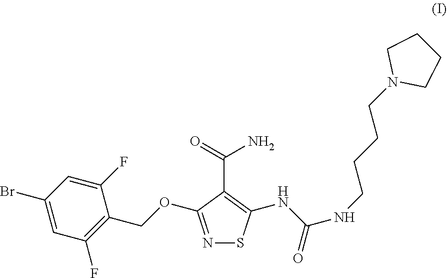 [SMILES: NC(=O)c1c(OCc2c(F)cc(Br)cc2F)nsc1NC(=O)NCCCCN1CCCC1]

or a pharmaceutically acceptable salt, prodrug, or solvate thereof, and a pharmaceutically acceptable carrier.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1A is a dose response curve showing that OSI-632 inhibited SMAD2/3 activity in IPF-LL29 cells. Error bars represent SEM of four replicates.

FIG. 1B is a series of plots showing time course changes in phosphorylation of SMAD2 (left) and ERK1/2 (right) in response to the indicated drug treatment.

FIG. 2A is a series of graphs showing that OSI-632 is a potent inhibitor of TGF-β-mediated IPF-lung myofibroblast activation. Plots show changes in TGF-β-induced expression of key myofibroblast activation markers and pro-fibrosis stimuli in the presence of indicated drug treatments in IPF-LL29 cells.

FIG. 2B is a series of graphs showing that OSI-632 is a potent inhibitor of TGF-β-mediated IPF-lung myofibroblast activation. Plots show changes in TGF-β-induced expression of key myofibroblast activation markers and pro-fibrosis stimuli in the presence of indicated drug treatments in and IPF-LL97A cells.

FIG. 3A is a series of bar graphs showing that OSI-632 treatment (1 mM) inhibited TGF-β-induced expression of COL1A1, ACTA2, and FN1 in human lung fibroblasts (left) and expression of indicated proliferation stimuli (right). Human lung fibroblasts (LL47) were treated with indicated drugs for 24 hours and gene expression was measured by qPCR.

FIG. 3B is a series of bar graphs showing that OSI-632 inhibited expression of indicated fibroblast activation and proliferation factors in mouse lung fibroblasts. Bars represent mean of triplicate and error bars represent SEM. * indicates p<0.05; ** indicates p<0.01; *** indicates p<0.05; **** indicates p<0.0001.

FIG. 4A is a plot showing changes in phosphorylation of ERK in response to time course FGF-basic stimulation in the presence of DMSO control or OSI-632.

FIG. 4B is a series of bar graphs showing changes in phosphorylation of indicated proteins in response to 30 min treatment of FGF basic (left), PDGF (middle) and VEGF (right).

FIG. 4C is a plot showing changes in phosphorylation of ERK in response to 30 min treatment of indicated growth factor stimulation in the presence of DMSO control or OSI-632.

FIG. 5A is a plot showing percent wound closure over 70 hours at different OSI-632 concentrations. OSI-632 potently decreased motility (i.e., reduced cell migration) of human lung fibroblasts from IPF patients in a dose-dependent manner in the real-time wound healing assay.

FIG. 5B is a plot showing 1 μM OSI-632 treatment significantly decreased TGF-β-stimulated contractility of IPF lung fibroblasts in collagen contraction assay.

FIG. 5C is a series of microscopy images showing OSI-632-mediated inhibition of motility and contraction, where OSI-632 significantly outperformed inhibition by nintedanib or pirfenidone (in a collagen contraction assay).

FIG. 6A is a schematic representation of a bleomycin-induced lung fibrosis model.

FIG. 6B is a series of representative Masson Trichrome images of lungs from untreated control mice (top), bleomycin-only treated mice (middle), bleomycin and OSI-632 treated mice (bottom). Arrows indicated collagen deposit as a proxy indicator of fibrosis. Intense collagen deposit and obliteration of lung architecture including septal thickening and collapse as well as irregular distribution of air spaces were observed in bleomycin treated mouse lungs. These symptoms were alleviated with OSI-632 treatment.

FIG. 6C is a bar graph showing the scoring of hematoxylin and eosin (H&E) and Masson Trichrome staining of lung tissue, showing that OSI-632 at 50 mg/kg significantly decreased lung fibrosis. *** indicates p<0.001.

FIG. 7A is a series of bar graphs showing OSI-632 treatment significantly reduced phosphorylation of indicated protein and total levels of collagen 1 in lung tissue collected from bleomycin induced IPF mouse model. Protein changes were measured using RPPA validated antibodies.

FIG. 7B is a series of representative immunohistochemistry (IHC) images and bar graphs showing that OSI-632 inhibits bleomycin-induced activation of STAT3. Representative IHC images showing reduction in nuclear intensity of activated STAT3 (Y705) in response to OSI-632 treatment (top); and plots show relative quantification of strong staining and H-score (bottom). Asterisk denotes statistical significance by one-way Anova compares to bleomycin treated group. * is p<0.05; ** p<0.01; *** p<0.001; **** p<0.0001. Bars represent means and error bars represent SEM.

FIG. 8 is a bar graph showing that OSI-632 inhibited activation of multiple key pro-fibrotic factors in vivo. Plots show changes in mRNA expression of indicated genes in fibrotic lung samples from bleomycin-induced lung fibrosis mouse model. Asterisk denotes statistical significance by one-way Anova compares to bleomycin treated group. * is p<0.05; ** p<0.01; *** p<0.001; **** p<0.0001. Bars represent means and error bars represent SEM.

FIG. 9A is a plot showing regression modeling to predict the response to >400 drugs, OSI-632 is among the top three predicted inhibitors.

FIG. 9B is a dose response curve showing OSI-632 inhibits SMAD2/3 activity in LX2 cells (left). Response to galunisertib is also shown. Error bars represent SEM of three replicates. Immunoblot showing OSI-632 blocks activation of TGF-β-mediated SMAD2 phosphorylation in LX2 cells (right).

FIGS. 10A-10C demonstrate that OSI-632 blocks hepatic stellate cell activation and function.

FIG. 10A shows that OSI-632 (1 mM) inhibited motility of LX2 cells measured by live cell wound healing assay. Response to galunisertib (Galu) is also shown. Bars represent means of three replicates and error bars represent SEM.

FIG. 10B is a bar graph showing that OSI-632 treatment (1 mM) inhibited TGF-β-induced expression of COL1A1 and αSMA in LX2 cells.

FIG. 10C is a series of bar graphs showing that OSI-632 inhibited expression of indicated collagens, HSC activation and proliferation factors.

FIGS. 11A-11C are microscopy images and bar graphs showing that OSI-632 reduced collagen deposition and promotes hepatocyte regeneration in vivo.

FIG. 11A are microscopy images (left) and bar graphs showing that OSI-632 treatment significantly reduces collagen staining. Plots showing quantification of percentage Masson Trichome staining at total, strong and moderate levels (center). Plots OSI-632 treatment decreases Col1a1 mRNA expression and protein levels (measured by RPPA) (right).

FIG. 11B is a bar graph showing that OSI-632 promoted hepatocyte regeneration indicated by Ki67 staining. Bar represent means and error bars represent SEM.

FIG. 11C is a series of bar graphs showing that OSI-632 inhibited CCl₄-induced expression of indicated ECM proteins, HSC activation markers and proliferation factors.

### DETAILED DESCRIPTION

The present disclosure is based on the discovery in a functional drug screen of hepatic myofibroblasts that a clinical grade compound of Formula (I) (also referred to herein as clinical grade compound 1 (Cpd1) or OSI-632) potently blocks TGF-β activity and significantly reverses the activated phenotypes of myofibroblast in vitro. The compound of Formula (I) can significantly reduce collagen deposition and stimulate hepatocyte regeneration. For example, treatment with OSI-632 can reverse expression of genes involved in ECM remodeling such as Timp1, Timp2, Timp3, and/or Loxl1; and reverse the expression of HSC activation marker (e.g., Acta2) and proliferation stimuli such as Tgfb2. Thus, as will be described in greater detail below, the compound of Formula (I) can inhibit ECM remodeling, collagen deposition, expression of HSC activation and proliferation stimuli, and promotes hepatocyte regeneration for the treatment of liver fibrosis. Furthermore, the effects of the compound of Formula (I) could be expanded to the treatment of fibrotic conditions in general, such as non-alcoholic steatohepatitis (NASH), cirrhosis, HBV infection, any liver disease, pulmonary fibrosis, interstitial lung disease, idiopathic pulmonary fibrosis (IPF), renal fibrosis, cardiac fibrosis, or any combination thereof.

It was discovered by the present inventors that the compound of Formula (I) can target multiple signaling pathways in fibrotic tissue in vivo and present advantages such as improved efficacy and reduction of drug resistance, compared to a therapeutic agent that targets a single signaling pathway. For example, treatment of mouse models of liver fibrosis produced increased levels of phosphorylation of Smad2 (a TGF-β downstream effector), Erk, pro-fibrosis growth factor receptors including Pdgfrb and Fgfr, and their downstream effectors including phosphorylated Src, Nfκβ, and STAT3. The in vivo effects of the compound of Formula (I) treatment are consistent with in vitro data.

Indeed, in vitro profiling of the compound of Formula (I) showed that it potently inhibits several growth factor receptors including FGFR and VEGFR as well as Src family kinases, which all have been implicated in myofibroblast activation.

As an example, in lung fibroblasts, the compound of Formula (I) is shown in the present disclosure to be a potent inhibitor of TGF-β signaling and TGF-β mediated release of ECM molecules and pro-fibrotic stimuli including IL11 and CTGF in lung fibroblast and IPF-lung fibroblast. Similar to models for liver fibrosis, OSI-632 can block downstream signaling of pro-fibrosis growth factors including TGF-β, FGF basic, PDGF, and/or VEGF in lung fibroblast and IPF-lung fibroblast; and can reverse activated phenotypes of lung fibroblast and IPF-lung fibroblast. In mammals, such as animal models, the compound of Formula (I) can significantly reduce collagen deposition, interstitial septal thickening, and collapse of air spaces in bleomycin-induced IPF; and can target multiple signaling pathways in the fibrotic lung. The compound of Formula (I) shows greater activity compared to existing approved IPF therapeutic agents, such as nintedanib or pirfenidone.

Advantageously, the compound of Formula (I) is believed to be safe and well-tolerated. The compound of Formula (I) was an oncology drug candidate that failed the phase 2 efficacy trial. The unique biology and tolerability of the compound of Formula (I) can enhance patient compliance and demonstrates its suitability for drug combination approach in conditions characterized by TGF-β activity, including excessive ECM deposition such as in fibrogenesis-related pathologies.

### Methods of Treatment

In accordance with the foregoing, in one aspect, the disclosure provides a method of inhibiting signaling mediated by binding of TGF-β, FGF, VEGF, and/or PDGF, to its respective receptor and subsequent activation, and/or blocking a Src family kinase in a cell. The method includes contacting the cell with a therapeutically effective amount of a compound with the structure set forth in the formula (I) below or a pharmaceutically acceptable salt, prodrug, or solvate thereof:

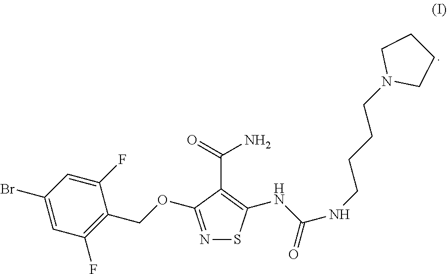 [SMILES: NC(=O)c1c(OCc2c(F)cc(Br)cc2F)nsc1NC(=O)NCCCCN1CCCC1]

This compound can otherwise be described as 3-(4-bromo-2,6-difluoro-benzyloxy)-5-[3-(4-pyrrolidin-1-yl-butyl)-ureido]-isothiazole-4-carboxylic acid amide, and is also referred to herein alternately as CP-547632, PAN-90806, OSI-632, and Cpd1. Additional description regarding the synthesis of the compound of formula (I), salts, prodrugs, and solvate thereof is provided below.

The cell referred to in the present disclosure encompasses any cell that produces extracellular matrix (ECM) proteins. For example, in response to injury, resident fibroblasts, pericytes, and other cell types in liver, lung, kidney or heart transdifferentiated into myofibroblasts or fibroblasts. The myofibroblasts express αSMA and have an increased capacity to produce large amounts of ECM proteins, leading to respective organ fibrosis. For proof of concept, the screening and in vitro results described below were initially performed using myofibroblasts transdifferentiated from hepatic stellate cells (HSCs), which are pericytes. While myofibroblasts and/or fibroblasts are accepted as a driver of fibrosis, it is believed that myofibroblasts and/or fibroblasts can be transdifferentiated from different cell types. In liver, HSCs are widely accepted as the major source. Accordingly, in some embodiments, the cell is a myofibroblast and/or a fibroblast. The myofibroblast and/or fibroblast can be derived from, or transdifferentiated from, any cell type. In some embodiments, the myofibroblast and/or fibroblast expresses αSMA. Exemplary myofibroblasts encompassed by the disclosure include liver myofibroblasts, lung myofibroblasts, kidney myofibroblast, and cardiac myofibroblasts. In one embodiment, the myofibroblast is a liver myofibroblast transdifferentiated from a hepatic stellate cell (HSC).

As described in more detail below, the disclosed compound of formula (I), was observed to interrupt (e.g., inhibit) signaling mediated by TGF-β, FGF, VEGF, PDGF, and Src family kinases. Accordingly, in one embodiment the compound inhibits TGF-β-mediated signaling in the cell. In one embodiment, the compound inhibits FGF-mediated signaling in the cell. In one embodiment, the compound inhibits VEGF-mediated signaling in the cell. In one embodiment, the compound inhibits PDGF-mediated signaling in the cell. In one embodiment, the compound inhibits Src family kinase-mediated signaling in the cell. Src family kinases are a group of non-receptor tyrosine kinases, all of which are contemplated by the present disclosure, including the SrcA subfamily (e.g., Src family kinase Src, Yes, Fyn, and Fgr) and the SrcB subfamily (Lck, Hck, Blk, and Lyn), and Frk in a third subfamily. In some embodiments, the compound inhibits two or more of the above signaling mediators, in any combination.

A person of ordinary skill in the art can readily test signaling modulation by the compound according to standard tests. Exemplary approaches are described in more detail below. In some embodiments, the inhibition of TGF-β, FGF, VEGF, PDGF, and/or a Src family kinase signaling in the cell by the compound results in reduced activation of SMAD2/3, Erk, and/or STAT3 in the cell. In some embodiments, the inhibition of TGF-β, FGF, VEGF, PDGF, and/or a Src family kinase signaling in the cell by the compound results in the inhibition of the phosphorylation and/or activation of of Src, Erk, JNK, S6, Nfκβ, STAT3, or any combination thereof.

In some embodiments, the inhibition of TGF-β, FGF, VEGF, PDGF, and/or a Src family kinase signaling in the cell by the compound results a decrease in the expression of TGF-β1, CTGF, IL-11, FGF, VEGF, FN1, or any combination thereof.

In some embodiments, the inhibition of TGF-β, FGF, VEGF, PDGF, and/or a Src family kinase signaling in the cell results in the inhibition of the expression of a downstream signaling protein mediated by TGF-β, FGF, VEGF, PDGF, and/or a Src family kinase. For example, the expression of αSMA, COL1A1, COL1A2, COL3A1, COL5A1, PDGFA, PDGFB, CTGF, IL11, VEGFA, CCL5, FN1, or any combination thereof can be inhibited.

In some embodiments, treatment with the compound of Formula (I) can reverse and/or inhibit expression of genes involved in ECM remodeling such as Timp1, Timp2, Timp3, and/or Loxl1.

In some embodiments, the cell is in an in vitro culture and the method comprises contacting (e.g., adding to) the cell with the compound of Formula (I) or a pharmaceutically acceptable salt, prodrug, or solvate thereof to the culture.

In some embodiments, the cell is in vivo in a subject with a condition treatable by inhibiting signaling mediated by TGF-β, FGF, VEGF, PDGF, and/or a Src family kinase. The method of these embodiments comprises administering to the subject a therapeutically effective amount of a pharmaceutical composition including: the compound of Formula (I) or a pharmaceutically acceptable salt, prodrug, or solvate thereof, and a pharmaceutically acceptable carrier. Elements regarding the formulation are described in more detail below.

Conditions treatable by inhibiting signaling mediated by TGF-β, FGF, VEGF, PDGF, and/or a Src family kinase include, but are not limited by, conditions characterized by excessive ECM proteins, including organ fibrosis, such as those described herein.

In another aspect, the disclosure provides a method of inhibiting activity of a myofibroblast and/or fibroblast. The method comprises contacting the myofibroblast and/or fibroblast with a therapeutically effective amount of a compound with the structure set forth in the formula (I) or a pharmaceutically acceptable salt, prodrug, or solvate thereof. Additional description regarding the synthesis of the compound of formula (I), salts, prodrugs, and solvate thereof is provided below.

The myofibroblast and/or fibroblast can be derived from, or transdifferentiated from, any cell type. In some embodiments, the myofibroblast and/or fibroblast expresses αSMA. Exemplary myofibroblasts encompassed by the disclosure include liver myofibroblasts, lung myofibroblasts, kidney myofibroblast, and cardiac myofibroblasts. In one embodiment, the myofibroblast is a liver myofibroblast is transdifferentiated from a hepatic stellate cell (HSC).

The term inhibiting activity of a myofibroblast encompasses reducing, and/or preventing phenotypes associated with an activated state of myofibroblasts. While such reduced activity includes reduction in production and/or deposition of extracellular matrix (ECM) molecules, reduction in expression and secretion of pro-fibrosis and pro-inflammatory stimuli by the myofibroblast, the reduced activity can also be characterized by other modulated phenotypes. For example, in some embodiments, inhibiting activity of a myofibroblast includes inhibiting contractility, proliferation, and/or motility of the myofibroblast, as can be determined by standard assays practiced in the art.

In some embodiments, the cell is in an in vitro culture. In such embodiments, the method comprises adding the compound or a pharmaceutically acceptable salt, prodrug, or solvate thereof to the culture.

In other embodiments, the cell is in vivo in a subject with a condition treatable by inhibiting activation of a myofibroblast and/or fibroblast. The method of these embodiments comprises administering to the subject a therapeutically effective amount of a pharmaceutical composition including: the compound or a pharmaceutically acceptable salt, prodrug, or solvate thereof, and a pharmaceutically acceptable carrier. Elements regarding the formulation are described in more detail below.

Conditions treatable by inhibiting activation of a myofibroblast include, but are not limited by, conditions characterized by excessive ECM proteins, including organ fibrosis, such as those described herein. Thus, in one exemplary embodiment, the subject has a fibrosis condition. In some embodiments, the subject has a liver disease, the liver disease can include liver fibrosis. In some exemplary embodiments, the fibrosis condition is, or is associated with, non-alcoholic steatohepatitis (NASH), cirrhosis, HBV infection, any liver disease, pulmonary fibrosis/interstitial lung disease, idiopathic pulmonary fibrosis (IPF), renal fibrosis, or cardiac fibrosis.

In another aspect, the disclosure provides a method of increasing hepatocyte regeneration in a subject in need thereof. The method comprises administering the subject with a therapeutically effective amount of a pharmaceutical composition comprising a compound with the structure set forth in the formula (I) or a pharmaceutically acceptable salt, prodrug, or solvate thereof. Additional description regarding the synthesis of the compound of formula (I), salts, prodrugs, and solvate thereof is provided below.

Increasing hepatocyte regeneration is beneficial in subjects with excessively active myofibroblast and/or fibroblast activation. For example, the method can be beneficial for a subject with a fibrosis condition of the liver. Exemplary fibrosis conditions can be, or be associated with, non-alcoholic steatohepatitis (NASH), cirrhosis, HBV infection, or any liver disease.

In some embodiments, the compound, or a pharmaceutically acceptable salt, prodrug, or solvate thereof inhibits collagen deposition, ECM remodeling, promotes hepatocyte regeneration, or a combination thereof, in a fibrotic liver tissue of the subject.

In another aspect, the disclosure provides a method of treating or preventing a fibrosis condition in a subject in need thereof. The method comprises administering the subject with a therapeutically effective amount of a pharmaceutical composition including a compound with the structure set forth in the formula (I), or a pharmaceutically acceptable salt, prodrug, or solvate thereof. Additional description regarding the synthesis of the compound of formula (I), salts, prodrugs, and solvate thereof is provided below.

In some embodiments, the method is a prophylactic method where the subject is not necessarily known to have any manifested fibrosis but may be at higher risk or likelihood of developing fibrosis. For example, based on medical history a subject may be more susceptible to developing fibrosis or have an enhanced likelihood of developing fibrosis. Such individuals may have sustained isolated or repeated injury to an organ or tissue, or may be infected with a virus (e.g., hepatitis) with an associated progression to fibrosis.

Alternatively, the subject has fibrosis and the method of treating comprises reducing, slowing, and/or reversing the fibrosis. A more expansive definition of “treating” is provided below.

In some embodiments, the fibrosis condition is, or is associated with, non-alcoholic steatohepatitis (NASH), cirrhosis, HBV infection, any liver disease, pulmonary fibrosis/interstitial lung disease, idiopathic pulmonary fibrosis (IPF), renal fibrosis, or cardiac fibrosis. In certain embodiments, the fibrosis condition is idiopathic pulmonary fibrosis, pulmonary fibrosis, and/or interstitial lung disease and the compound inhibits collagen deposition, interstitial septal thickening, collapse of air spaces, inflammation, or any combination thereof, in a pulmonary fibrotic tissue.

In some embodiments, the method includes administering the subject with a therapeutically effective amount of a second pharmaceutical composition to treat fibrosis. In such combination therapies, the first pharmaceutical composition can be administered before, simultaneously with, or after administering the second pharmaceutical composition. The second pharmaceutical composition can be any pharmaceutical agent (e.g., any chemical or biological agent) that is known or used to treat fibrosis or related conditions. For example, in some embodiments, the second composition includes nintedanib, pirfenidone, GLPF1690, pamreclumab, obeticholic acid (OCA), elafabranor, and/or cenicriviroc.

The first pharmaceutical composition and/or the second pharmaceutical composition can independently be administered multiple times (e.g., 2 times, 3 times, 4 times, 5 times, 6 times, 7 times, 8 times, 9 times, or 10 times) or continuously over a given time period (e.g., a day, 2 days, 4 days, a week, or a month). For example, the first pharmaceutical composition and/or the second pharmaceutical composition can independently be administered. In some embodiments, the first pharmaceutical composition and the second pharmaceutical composition can be administered daily. In some embodiments, the first pharmaceutical composition can be administered daily, while a second pharmaceutical composition can be administered on a weekly basis.

### Compound

This section provides additional description of the compound of Formula (I), (OSI-632, also referred to as CP-547632, PAN-90806, OSI-632, and 3-(4-bromo-2,6-difluoro-benzyloxy)-5-[3-(4-pyrrolidin-1-yl-butyl)-ureido]-isothiazole-4-carboxylic acid amide). This compound, and exemplary methods of making it, are described in Larson, et al., U.S. Pat. No. 6,235,764, incorporated herein by reference in its entirety.

Methods of preparing the pharmaceutical composition with a specific amount of active compound are known, or will be apparent, to those skilled in this art. For example, see Remington's Pharmaceutical Sciences, Mack Publishing Company, Easter, Pa., 15th Edition (1975). In one embodiment, the 3-(4-Bromo-2,6-difluoro-benzyloxy)-5-[3-(4-pyrrolidin-1-yl-butyl)-ureido]-isothiazole-4-carboxylic acid amide can be prepared from [3-(4-bromo-2,6-difluoro-benzyloxy)4-carbamoyl-isothiazol-5-yl]-carbamic acid phenyl ester and 4-pyrrolidin-1-yl-butylamine by the procedure analogous to Example 1 of the '764 patent, incorporated herein by reference in its entirety.

As used herein and unless otherwise indicated, the phrase “pharmaceutically acceptable salt” includes salts of acidic or basic groups which can be present in the compound. Basic groups are capable of forming a wide range of salts with various inorganic and organic acids. Nonlimiting acids that can be used to prepare pharmaceutically acceptable acid addition salts are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. In some embodiments, the pharmaceutically acceptable salt of the compound is a hydrochloride salt or a mesylate salt of the compound of formula (I).

Generally, prodrugs can be created by adding to free amino, amido, hydroxy or carboxylic groups in a compound. Exemplary prodrugs include compounds wherein an amino acid residue, or a polypeptide chain of two or more (e.g., two, three or four) amino acid residues is covalently joined through an amide or ester bond to a free amino, hydroxy or carboxylic acid of the compound. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs also encompassed include ones where free carboxyl groups can be derivatized as amides or alkyl esters. The amide and ester moieties may incorporate groups including but not limited to ether, amine and carboxylic acid functionalities. Free hydroxy groups may be derivatized using groups including but not limited to hemisuccinates, phosphate esters, dimethylaminoacetates, and phosphoryloxymethyloxycarbonyls, as outlined in D. Fleisher, et al., Advanced Drug Delivery Reviews (1996) 19:115. Carbamate prodrugs of hydroxy and amino groups are also included, as are carbonate prodrugs and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in R. P. Robinson et al., J. Medicinal Chemistry (1996) 39:10. Method of preparing prodrugs are also described, for example, in V. K. Redasani and S. B. Bari, Prodrug Design: Perspectives, Approaches and Applications in medicinal Chemistry, Academic Press; 1st edition (Jul. 28, 2015), incorporated herein by reference in its entirety.

Administration of the disclosed compound of Formula (I) can be accomplished by any method that enables delivery of the compounds to the target organ or tissue. These methods include oral routes, intraduodenal routes, parenteral injection (including intravenous, subcutaneous, intramuscular, intravascular, and/or infusion), topical, and rectal administration. In some embodiments, administration of the compound of Formula (I) is by an oral route.

The dosing of the active compound (e.g., a compound of Formula (I), or a pharmaceutically acceptable salt, prodrug, or solvate thereof) administered will be dependent on the subject being treated, the severity of the disorder or condition, the rate of administration and the judgement of the prescribing physician. For example, a therapeutically effective dosage can be a dosage equivalent to the compound of Formula (I) in the range of about 0.001 to about 100 mg per kg body weight per day. For example, the compound, or a pharmaceutically acceptable salt, prodrug, or solvate thereof, can be administered at a dosage equivalent to the compound of Formula (I) of from 10 mg/kg (e.g., from 20 mg/kg, from 30 mg/kg, from 40 mg/kg, from 50 mg/kg, from 60 mg/kg, from 70 mg/kg, from 80 mg/kg, or from 90 mg/kg) to 100 mg/kg (e.g., to 90 mg/kg, to 80 mg/kg, to 70 mg/kg, to 60 mg/kg, to 50 mg/kg, to 40 mg/kg, to 30 mg/kg, or to 20 mg/kg) per day. In certain embodiments, the dosage equivalent to the compound of Formula (I) is from about 1 mg/kg/day (e.g., from about 10 mg/kg/day, from 30 mg/kg/day, from 40 mg/kg/day, from 50 mg/kg/day, from 60 mg/kg/day, from 70 mg/kg/day, from 80 mg/kg/day, or from 90 mg/kg/day) to about 100 mg/kg/day (e.g., to 90 mg/kg/day, to 80 mg/kg/day, to 70 mg/kg/day, to 60 mg/kg/day, to 50 mg/kg/day, to 40 mg/kg/day, to 30 mg/kg/day, to 20 mg/kg/day, or to 10 mg/kg/day), in single or divided doses. In some instances, dosage levels below the lower limit of the aforesaid range can be more than adequate, while in other cases still larger doses can be used without causing any harmful side effect, with the caveat that such larger doses may require splitting into several small doses for appropriately timed administration.

The pharmaceutical composition can be formulated for any suitable administration method. For example, the composition can be presented in a form suitable for oral administration as a tablet, capsule, pill, powder, sustained release formulations, solution, suspension, for parenteral injection as a sterile solution, suspension or emulsion, for topical administration as an ointment or cream, or for rectal administration as a suppository. The pharmaceutical composition can be in unit dosage forms suitable for single administration of precise dosages. The pharmaceutical composition typically includes a conventional pharmaceutical carrier or excipient and a compound according to the invention as an active ingredient. In some embodiments, the formulation includes additional pharmaceutical agents (e.g., second pharmaceutical agent). Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. In some embodiments, the pharmaceutical compositions can include additional ingredients such as flavorings, binders, excipients and the like. For example, for oral administration, tablets can contain various excipients, such as citric acid, together with various disintegrants such as starch, alginic acid and certain complex silicates and with binding agents such as sucrose, gelatin and acacia. Additionally, lubricating agents, such as magnesium stearate, sodium lauryl sulfate and talc, can be incorporated for tableting purposes. Solid compositions of a similar type can also be employed in soft and hard filled gelatin capsules. In embodiments of aqueous suspensions or elixirs oral administration, the active compound therein can be combined with various sweetening or flavoring agents, coloring matters or dyes and, if desired, emulsifying agents or suspending agents, together with diluents such as water, ethanol, propylene glycol, glycerin, or combinations thereof.

### Additional Definitions

Unless specifically defined herein, all terms used herein have the same meaning as they would to one skilled in the art of the present invention. Practitioners are particularly directed to Sambrook J., et al., (eds.), Molecular Cloning: A Laboratory Manual, 3rd ed., Cold Spring Harbor Press, Plainsview, New York (2001); Ausubel, F. M., et al., (eds.), Current Protocols in Molecular Biology, John Wiley & Sons, New York (2010); Coligan, J. E., et al., (eds.), Current Protocols in Immunology, John Wiley & Sons, New York (2010); Meyer K. C. & Stevens, S. D. (eds.), Idopathic Pulmonary Fibrosis: A Comprehensive Clinical Guide, Respiratory Medicine, Springer Nature Switzerland, (2019); and Friedman, S. S. & Martin, P., Handbook of Liver Disease, Elsevier (2017) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The terms “subject,” “individual,” and “patient” are used interchangeably herein to refer to a mammal being assessed for treatment and/or being treated. In certain embodiments, the mammal is a human. The terms “subject,” “individual,” and “patient” encompass, without limitation, individuals having fibrosis. While subjects may be human, the term also encompasses other mammals, particularly those mammals useful as laboratory models for human disease, e.g., mouse, rat, dog, non-human primate, and the like.

The term “treating” and grammatical variants thereof may refer to any indicia of success in the treatment or amelioration or prevention of a disease or condition (e.g., a fibrosis-related disease such as NASH or IPF), including any objective or subjective parameter such as abatement; remission; diminishing of symptoms or making the disease condition more tolerable to the patient; slowing in the rate of degeneration or decline; or making the final point of degeneration less debilitating.

The treatment or amelioration of symptoms can be based on objective or subjective parameters; including the results of an examination by a physician. Accordingly, the term “treating” includes the administration of the compounds or agents of the present disclosure to prevent or delay, to alleviate, or to arrest or inhibit development of the symptoms or conditions associated with disease or condition (e.g., a fibrosis-related disease such as NASH or IPF). The term “therapeutic effect” refers to the reduction, elimination, or prevention of the disease or condition, symptoms of the disease or condition, or side effects of the disease or condition in the subject.

As used herein, the phrase “therapeutically effective amount” refers to the amount of a therapeutic agent (i.e., drug, or therapeutic agent composition) that elicits the biological or medicinal response that is being sought in a tissue, system, animal, individual or human by a researcher, veterinarian, medical doctor or other clinician, which includes one or more of the following:
(1) preventing the disease; for example, preventing a disease, condition or disorder in an individual who may be predisposed to the disease, condition or disorder but does not yet experience or display the pathology or symptomatology of the disease;
(2) inhibiting the disease; for example, inhibiting a disease, condition or disorder in an individual who is experiencing or displaying the pathology or symptomatology of the disease, condition or disorder; and
(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES. As used herein, with respect to measurements, “about” means +/−5%. As used herein, a recited ranges includes the end points, such that from 0.5 mole percent to 99.5 mole percent includes both 0.5 mole percent and 99.5 mole percent.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1. Idiopathic Pulmonary Fibrosis Model

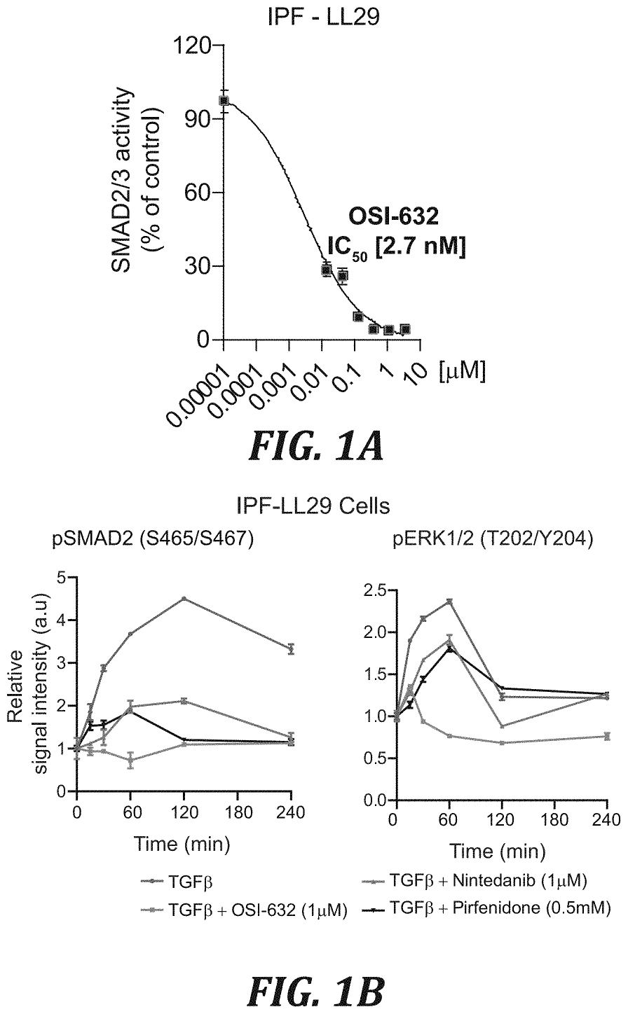

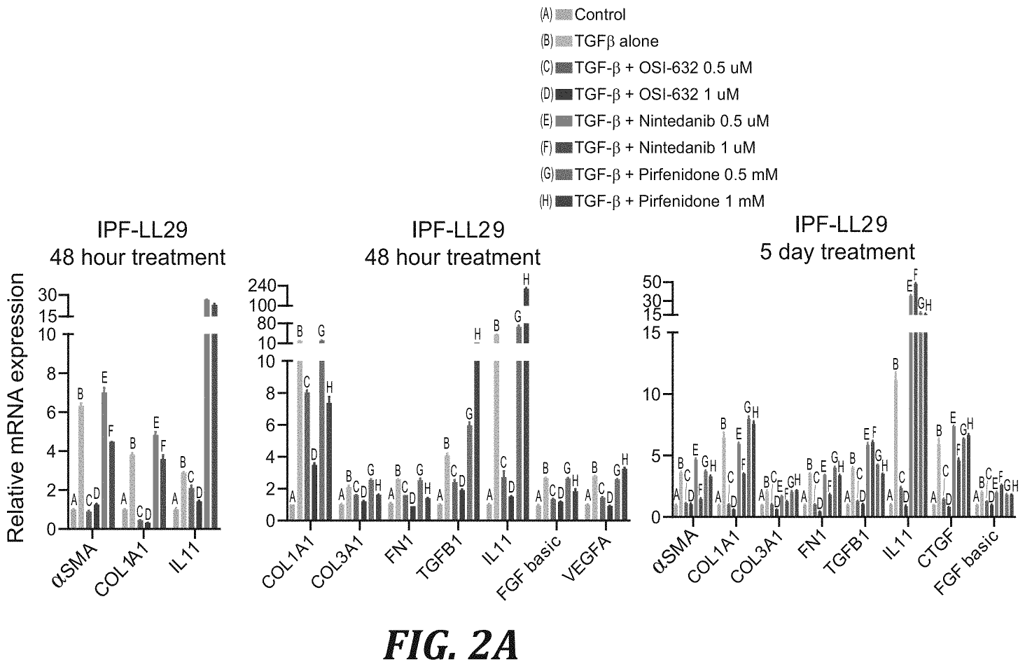

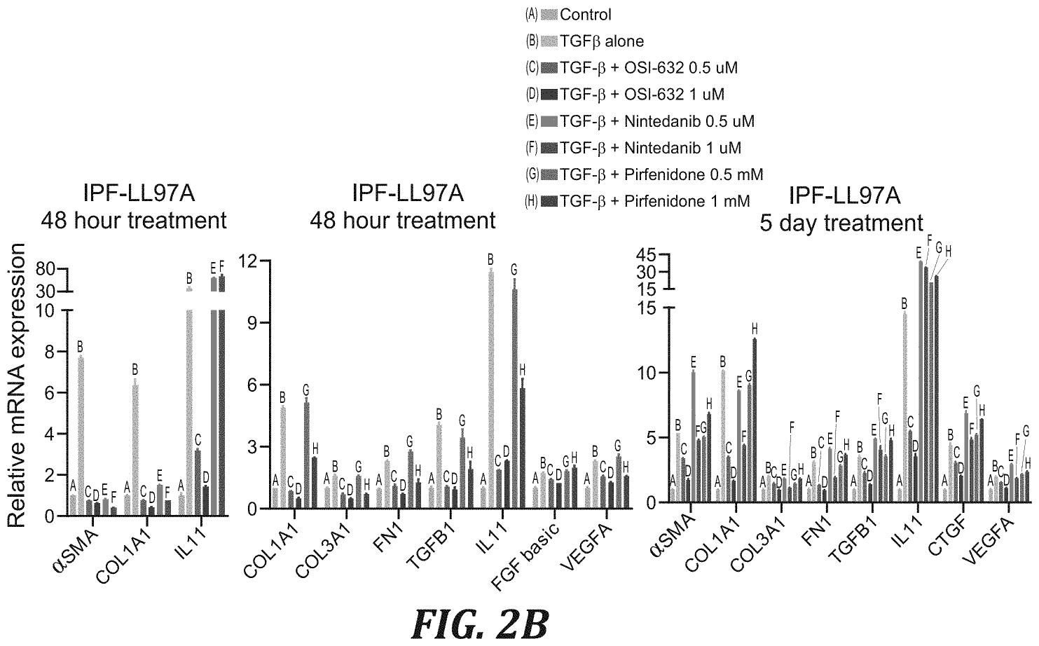

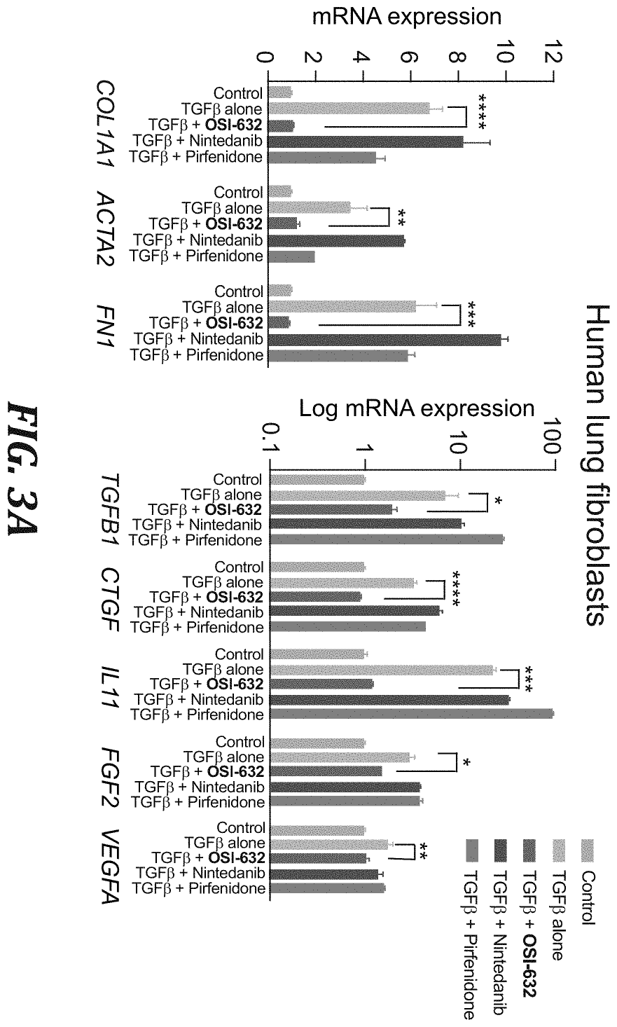

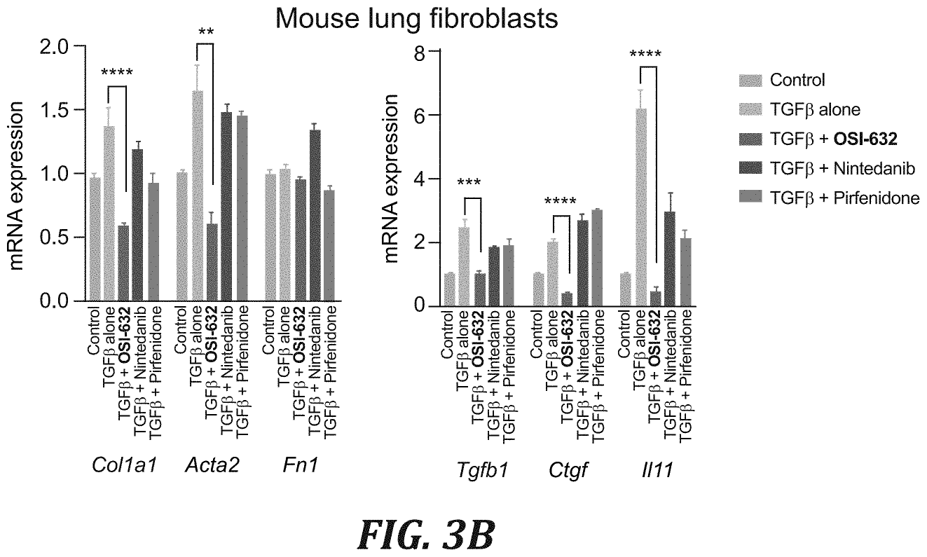

The present Example demonstrates that OSI-632 is a potent inhibitor of TGF-β signaling in IPF-lung fibroblast and blocks activation of lung myofibroblasts. Referring to FIGS. 1A and 1B, 1 mM OSI-632 treatment significantly decreased activity of TGF-β (e.g., as measured by phosphorylation of SMAD2/3 and Erk). In addition, referring to FIGS. 2A and 2B, OSI-632 treatment decreased TGF-β-induced expression of myofibroblast activation markers such as αSMA, COL1A1, COL3A1 and FN1 in human lung fibroblast cell lines derived from IPF patients. Referring again to FIGS. 2A and 2B, a significant decrease in the expression of myofibroblast proliferation stimuli such as TGFB1, CTGF, IL-11, FGF2 and VEGFA was observed. Referring to FIGS. 3A and 3B, similar results were observed in TGF-β-activated primary mouse lung fibroblasts and human lung fibroblast cell line derived from healthy donor. Referring to FIGS. 2A, 2B, 3A, and 3B, treatment with nintedanib at a same equivalent dose or pirfenidone at a much higher dose than the equivalent dose did not effectively block TGF-β induced expression of myofibroblast activation marker genes or proliferation stimuli. Together, these data demonstrate that OSI-632 inhibited lung fibroblast activation and outperformed existing currently approved IPF drugs.

### OSI-632 Mechanism of Action

OSI-632 as an Inhibitor of TGF-β

IPF-LL29, IPF-LL97A, and human lung fibroblast LL47 cell lines were purchased from ATCC. Mouse lung fibroblasts were isolated from the lungs of male C57BL/6J mice. Cells were grown at 37° C. under 5% CO2, 95% ambient atmosphere and maintained in Dulbecco's minimum essential medium (DMEM) supplemented with 10% FBS (Sigma) and 1% Penn Strep.

Methods:

FIG. 1A. A TGF-β reporter cell line was generated by transducing a SMAD firefly luciferase reporter (Cellomics Tech, Cat #PLV-10099-50) into IPF-LL29 cells. TGF-β at 5 ng/ml and OSI-632 at various concentrations were added to these cells for 24 hour. 24 hours later, luciferase substrate was added to the cells and luciferase signal was measured by Bright-Glo reagent (Promega) using a luminescent plate reader. The intensity of the signal correlates with the activity of SMAD2/3.

FIG. 1B. IPF-LL29 cells were incubated with TGF-β alone at 5 ng/ml or TGF-β with OSI-632 at 1 μM or Nintedanib at 1 μM or Pirfenidone at 0.5 mM for 0 min, 10 min, 30 min, 1 hour, 2 hour and 4 hour. At the end of the experiment, cells were lysed in 2% SDS lysis buffer. Protein levels of phosphorylated SMAD2 and ERK were detected in the cell lysates by reverse-phase protein array.

FIGS. 2A and 2B. IPF-29 (FIG. 2A) or IPF-97A (FIG. 2B) cells were incubated with TGF-β alone at 5 ng/ml or TGF-β with indicated drugs for 48 hours and 5 days. Total cellular RNA was isolated using a RNeasy Mini Kit (QIAGEN). 1 μg of total RNA was reverse transcribed into first-strand cDNA using an RT2 First Strand Kit (QIAGEN). The resultant cDNA was subjected to qPCR using indicated human primers to measure the changes in mRNA expression of indicated human genes.

FIGS. 3A and 3B. Human lung fibroblast LL47 and mouse primary lung fibroblasts isolated from mouse lungs were incubated with TGF-β alone at 5 ng/ml or TGF-β with indicated drugs for 24 hours. Same method as outlined in FIGS. 2A and 2B was used to measure the mRNA expression of indicated human or mouse genes.

FIGS. 1A and 1B show that OSI-632 inhibited TGF-β induced downstream signaling in IPF-lung fibroblasts. FIG. 1A is a dose response curve showing that OSI-632 inhibited SMAD2/3 activity in IPF-LL29 cells. Error bars represent SEM of four replicates. FIG. 1B is a series of plots showing time course changes in phosphorylation of SMAD2 (Left) and ERK1/2 (Right) in response to the indicated drug treatment.

FIGS. 2A and 2B are a series of graphs showing that OSI-632 is a potent inhibitor of TGF-β mediated IPF-lung myofibroblast activation. Plots show changes in TGF-β induced expression of key myofibroblast activation markers and pro-fibrosis stimuli in the presence of indicated drug treatments in IPF-LL29 cells (FIG. 2A) and IPF-LL97A cells (FIG. 2B).

FIGS. 3A and 3B demonstrate that OSI-632 is a potent inhibitor of TGF-β mediated lung myofibroblast activation. FIG. 3A is a series of bar graphs showing that OSI-632 treatment (1 mM) inhibited TGFb-induced expression of COL1A1, ACTA2, and FN1 in human lung fibroblasts (left) and expression of indicated proliferation stimuli (right). Human lung fibroblasts (LL47) were treated with indicated drugs for 24 hours and gene expression was measured by qPCR. FIG. 3B is a series of bar graphs showing that OSI-632 inhibited expression of indicated fibroblast activation and proliferation factors in mouse lung fibroblasts. Bars represent mean of triplicate and error bars represent SEM. * indicates p<0.05; ** indicates p<0.01; *** indicates p<0.05; **** indicates p<0.0001.

OSI-632 as an Inhibitor of FGF Basic, VEGF and PDGF Signaling

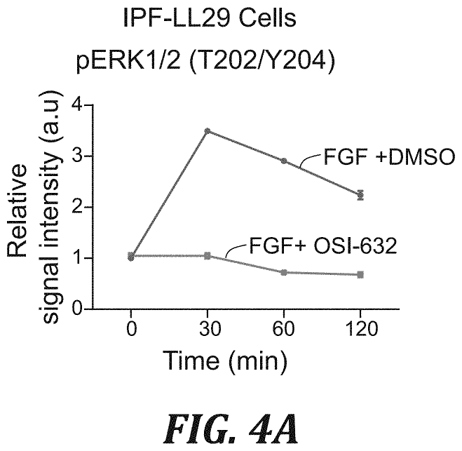

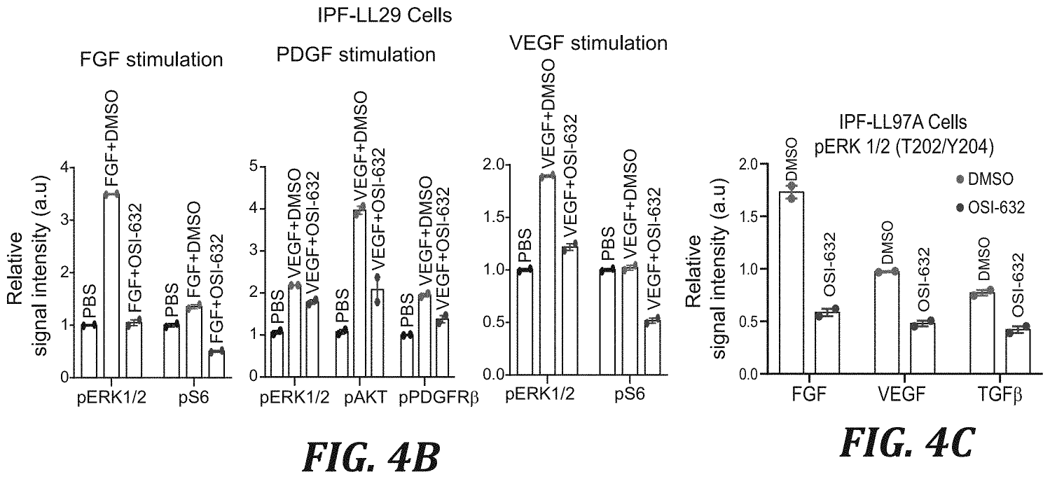

FIG. 4A. IPF-LL29 cells were incubated with FGF basic alone at 20 ng/ml or together with OSI-632 at 1 μM for 0 min, 30 min, 1 hour and 2 hour. At the end of the experiment, cells were lysed in 2% SDS lysis buffer. Protein levels of phosphorylated ERK were detected in the cell lysates by reverse-phase protein array.

FIG. 4B. IPF-LL29 cells were incubated with indicated growth factor alone or together with OSI-632 at 1 μM for 30 min. At the end of the experiment, cells were lysed in 2% SDS lysis buffer. Changes in the phosphorylation levels of indicated proteins were detected in the cell lysates by reverse-phase protein array.

FIG. 4C. IPF-LL97A cells were incubated with indicated growth factor alone or together with OSI-632 at 1 μM for 30 min. At the end of the experiment, cells were lysed in 2% SDS lysis buffer. Changes in phosphorylated ERK (pERK1/2) were detected in the cell lysates by reverse-phase protein array.

OSI-632 is a potent inhibitor of FGF basic, VEGF and PDGF signaling in IPF-lung fibroblast. The data show that 1 mM OSI-632 treatment significantly decreased phosphorylation of multiple growth factor induced signaling proteins including ERK1/2, AKT, S6 ribosome protein and PDGFRb in two different IPF-lung fibroblast cell lines.

FIGS. 4A-4C show that OSI-632 was a potent inhibitor of multiple growth factor signaling pathways in IPF-lung fibroblasts. FIG. 4A is a plot showing changes in phosphorylation of ERK in response to time course FGF basic stimulation in the presence of DMSO control or OSI-632. FIG. 4B is a series of bar graphs showing changes in phosphorylation of indicated proteins in response to 30 min treatment of FGF basic (Left), PDGF (Middle) and VEGF (Right). FIG. 4C is a plot showing changes in phosphorylation of ERK in response to 30 min treatment of indicated growth factor stimulation in the presence of DMSO control or OSI-632.

OSI-632 Inhibition of Fibroblast Motility and Contractility

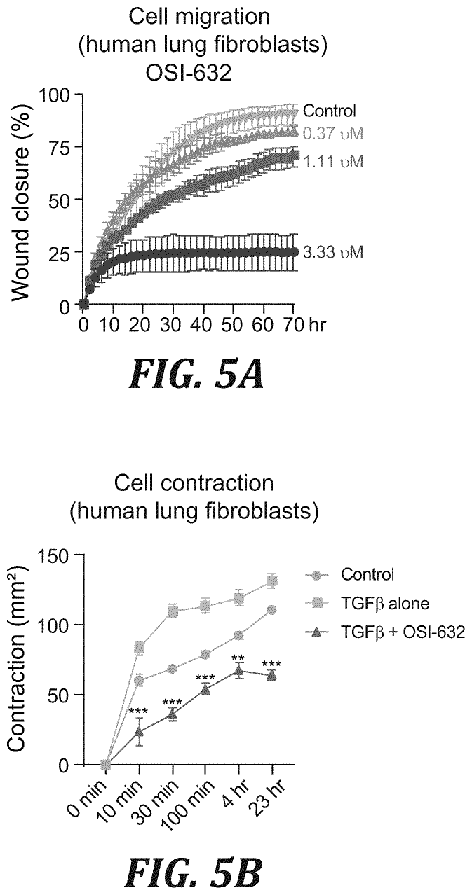

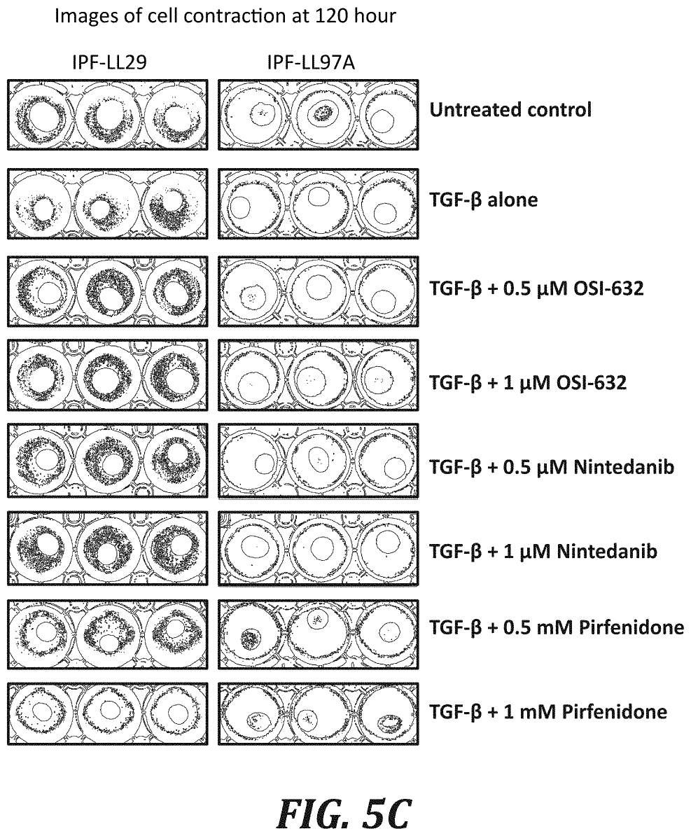

FIG. 5A: LL47 cells were plated on 96-well plates (Essen Image Lock, Essen Instruments). 24 hours later, OSI-632 at various concentrations was added and a wound was scratched with wound scratcher (Essen Instruments). Wound closure was monitored with Incucyte Live-Cell Imaging System and software (Essen Instruments) every 2-4 hours for 72 hours. The mean relative wound density of at least three biological replicates was calculated. The wound density correlates with the cell motility.

FIG. 5B: LL-47 cells were cultured in a collagen gel solution in 24-well plate. 24 hours later, TGF-β alone or together with OSI-632 was added. After 24 hours, cell contraction was initiated by releasing the collagen gels from the sides of the culture dishes. The collagen gel size change (contraction index) was measured at various times and quantified with Image Lab.

FIG. 5C: same method as outlined in FIG. 5B. IPF-LL29 and IPF-LL97A cells were cultured in a collagen gel solution. 24 hours later, TGF-β alone or together with indicated drugs was added for 24 hours before cell contraction was initiated. Images shown here were taken 120 hours after the initiation of cell contraction.

FIGS. 5A-5C show that OSI-632 blocked cell motility and contractility in lung fibroblast cell lines derived from IPF patients. Referring to FIG. 5A, increased cell motility and contractility are the major hallmarks of activated myofibroblasts. The data show that OSI-632 potently decreased motility (i.e., reduced cell migration) of human lung fibroblasts from IPF patients in a dose-dependent manner in a real-time wound healing assay. OSI-632 treatment significantly reduces cell migration in a real-time wound healing assay. FIG. 5B consistently showed 0.5 or 1 mM OSI-632 treatment also significantly decreased contractility of IPF lung fibroblasts in collagen contraction assay. Importantly, referring to FIG. 5C, data also showed that OSI-632-mediated inhibition of motility and contraction significantly outperforms inhibition by nintedanib or pirfenidone (in a collagen contraction assay). Together, these compelling data establish that OSI-632 inhibited TGF-β stimulated lung fibroblast activation and function.

### Animal Studies

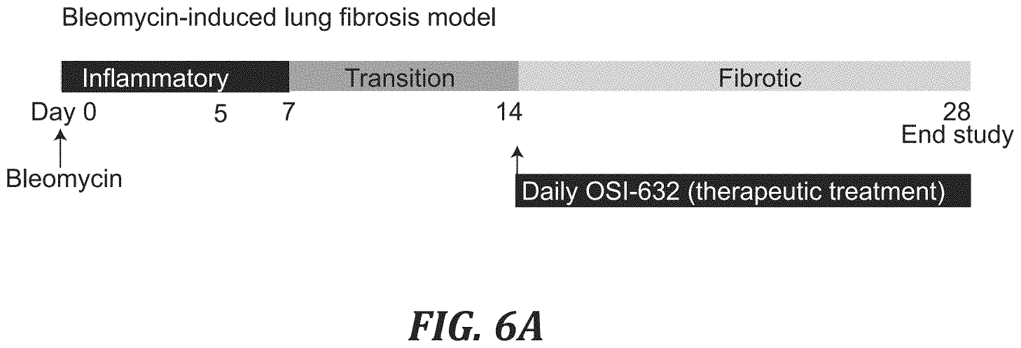

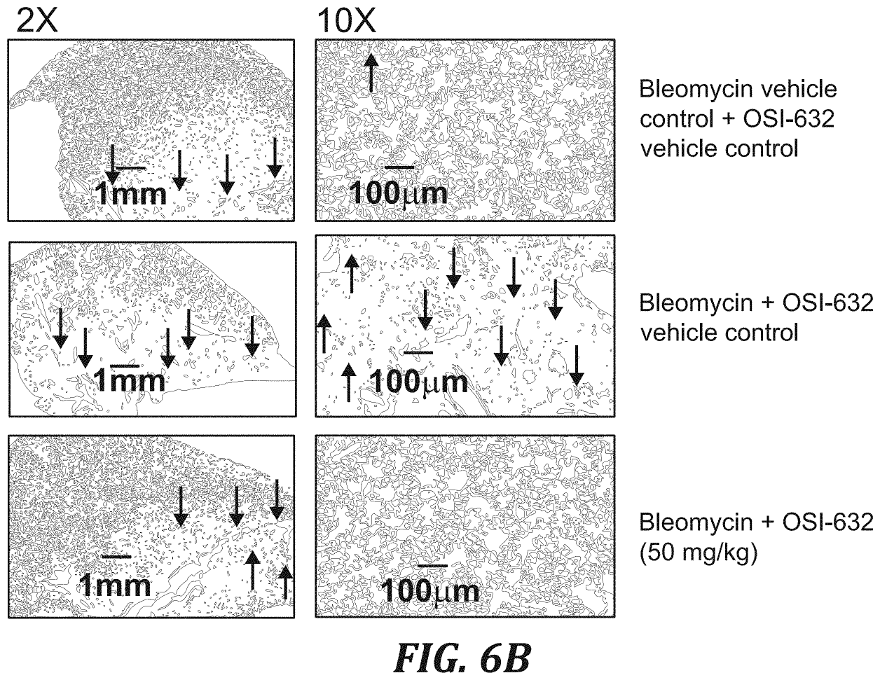

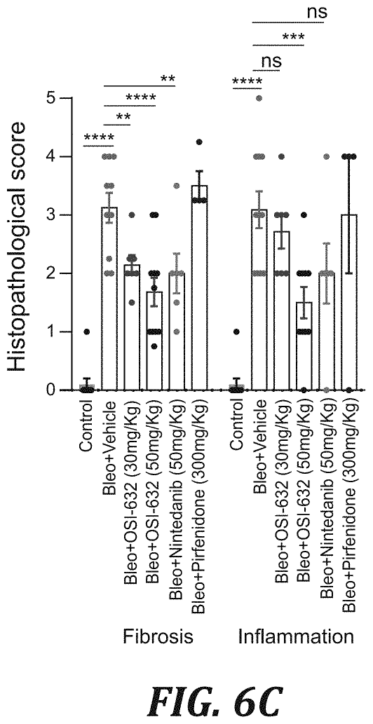

FIG. 6B: Male C57BL/6J mice between 7-10 week old were used in the study. A single dose of 50 IU of bleomycin was given to mouse lung via oropharyngeal aspiration to induce lung fibrosis. 14 days later, mice were treated orally with OSI-632 molecule from Monday to Friday once a day for 2 weeks. Mice were sacrificed on Day 28 after bleomycin treatment. Lungs were either snap frozen or fixed in 10% formalin and stained for H&E and Masson Trichrome.

FIG. 6C: H&E and Masson Trichrome staining were evaluated and scored by a board-certified veterinary pathologist.

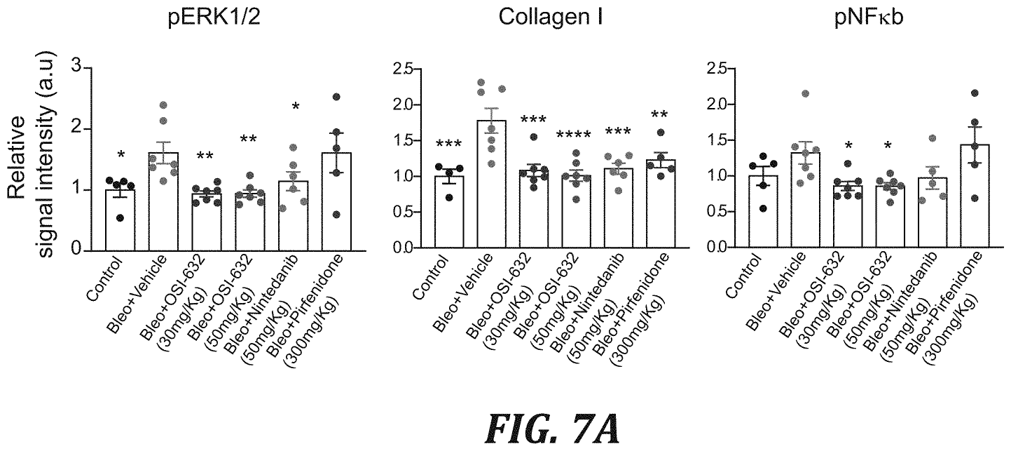

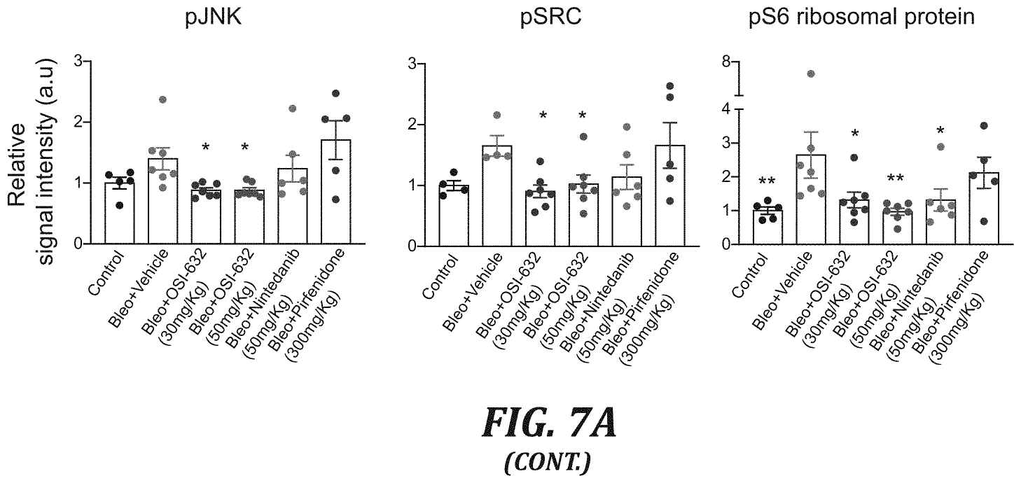

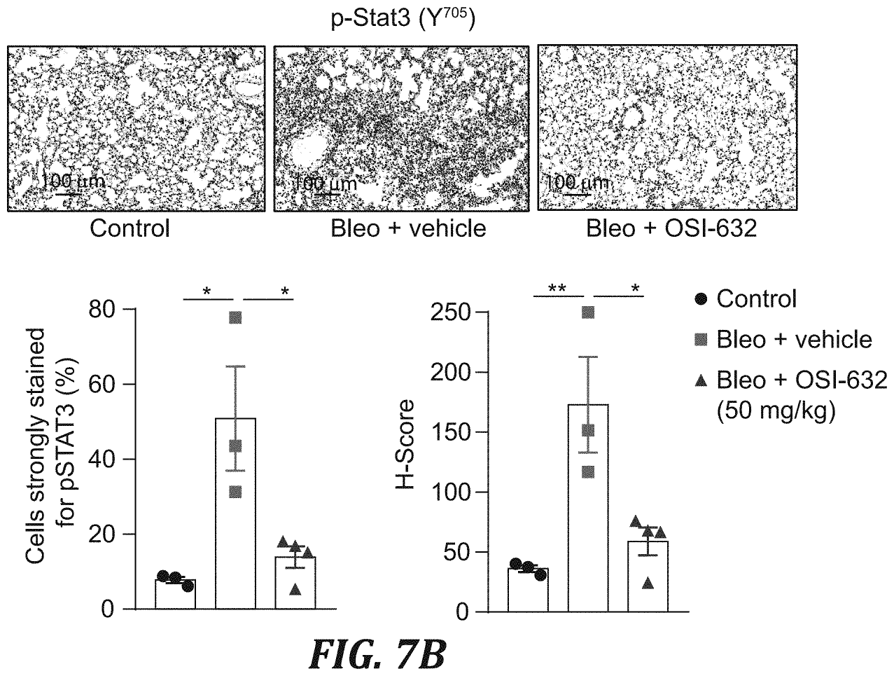

FIG. 7A: Total protein was extracted from the snap frozen lung tissue in T-PER™ Tissue Protein Extraction Reagent by Omni Bead Ruptor homogenizer. Changes in collagen I and the phosphorylation levels of indicated proteins were detected in the lung homogenates from the bleomycin in vivo studies by reverse-phase protein array.

FIG. 7B: pSTAT3 levels were detected in the fixed lung tissue by immunohistochemistry. Intensity of staining was quantified by HALO imaging software.

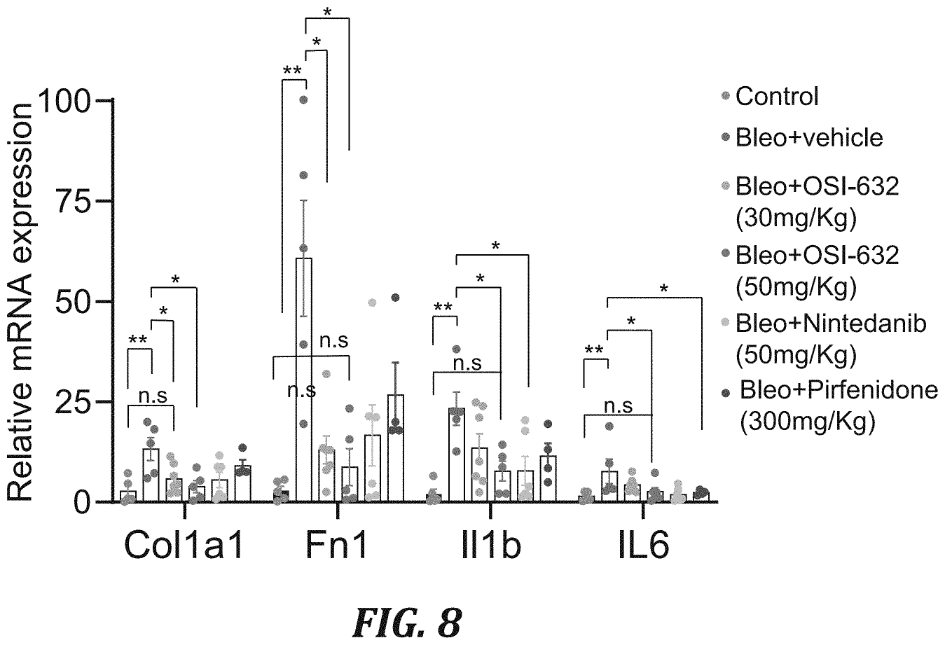

FIG. 8: Total RNA was extracted from the snap frozen lung tissue in TRIzol™ Reagent by Omni Bead Ruptor homogenizer. 1 μg of total RNA was reverse transcribed into first-strand cDNA using an RT2 First Strand Kit (QIAGEN). The resultant cDNA was subjected to qPCR using indicated mouse primers to measure the changes in mRNA expression of indicated mouse genes.

In this assay, whether OSI-632 could inhibit lung fibrosis in mice was investigated. OSI-632 significantly reduced collagen deposition in bleomycin-induced mouse model of IPF.

Animal Model

Without wishing to be bound by theory, it is believed that there are no known natural models for IPF. While no animal model can recapitulate all features of human disease, the bleomycin-induced mouse model is the best-characterized and currently most extensively used animal model due to its ability to reproduce many aspects of IPF and lung fibrosis associated with other interstitial lung diseases, ease of use, and short timeframe to developing fibrosis (FIG. 6A).

Bleomycin is an antibiotic primarily for cancer treatment with pulmonary fibrosis as its major adverse effect due to the lack of bleomycin-inactivating enzyme, bleomycin hydrolase, in lung. It has been established as an agent to induce experimental lung injury in different animal species including mice. This model exhibits some molecular signatures and histopathological hallmarks of lung fibrosis that resemble those observed in IPF, including TGF-β activation, alveolar epithelial cell injury and basement membrane damage, subsequent release of pro-inflammatory and pro-fibrotic mediators, followed by excessive extracellular matrix deposition in interstitial and intra-alveolar spaces by activated fibroblasts and myofibroblasts which eventually results in loss of normal lung architecture. Particularly, it is the most applicable model to evaluate novel therapies for treating IPF. Both nintedanib and pirfenidone, the only approved therapies for IPF, utilized bleomycin model in their pre-clinical assessment.

Reduction in Collagen Deposition, Interstitial Septal Thickening and Collapse of Air Spaces

Treatment with OSI-632 (50 mg/kg) caused a significant decrease in bleomycin-induced lung fibrosis (FIGS. 6B and 6C). Masson trichome staining showed significant reduction in collagen deposition in OSI-632 treated group compared with bleomycin only treated mice. OSI-632 at 50 mg/kg significantly decreased lung fibrosis measured by scoring of H&E and Masson Trichrome staining by veterinary pathologist. Importantly, treatment with OSI-632 did not result in mortality (100% overall survival).

Referring to FIG. 6A-6B, OSI-632 treatment alleviated lung fibrosis in bleomycin-induced mouse model of IPF. A single dose of 25-50 IU of bleomycin was given to mouse lung via oropharyngeal aspiration to induce lung fibrosis. 14 days later, mice were treated orally with OSI-632 from Monday to Friday once a day for 2 weeks Mice were sacrificed on Day 28 after bleomycin treatment. Lungs were processed and stained for H&E and Masson Trichrome.

FIG. 6A shows a schematic representation of the bleomycin model. Referring to FIG. 6B, representative Masson Trichrome images of lungs from untreated control mice (top), bleomycin-only treated mice (middle), bleomycin and OSI-632 treated mice (bottom) are shown. Arrows indicated collagen deposit as a proxy indicator of fibrosis. Intense collagen deposit and obliteration of lung architecture including septal thickening and collapse as well as irregular distribution of air spaces were observed in bleomycin treated mouse lungs. These symptoms were alleviated with OSI-632 treatment. Referring to FIG. 6C, OSI-632 significantly decreased lung fibrosis and inflammation as measured by scoring of H&E and Masson Trichrome staining by veterinary pathologist. **, ***, **** indicate p<0.01, 0.001 and 0.0001, respectively.

OSI-632 targets multiple signaling pathways in the fibrotic lung OSI-632 target engagement was validated in vivo. Referring to FIGS. 7A and 7B, molecular analyses of bleomycin-treated mouse lungs showed increased levels of phosphorylation of downstream effectors of pro-fibrosis growth factors including phosphorylated Src, Erk, JNK, S6 and NFκβ compared with vehicle treated control cohort. Consistent with the Masson Trichrome staining, bleomycin-treated lungs showed increased collagen protein levels (see, FIG. 6C). In agreement with in vitro IPF-lung fibroblast activation data (FIGS. 1A, 1B, 4A, 4B, and 4C), OSI-632 treatment significantly decreased bleomycin-induced collagen levels and other key pro-fibrotic factors including Fn1, IL-6 and IL-1b (FIG. 8) as well as phosphorylation of Src, Erk, JNK, S6, Nfκβ, and pSTAT3 compared with bleomycin-only treated mouse lung tissues (FIGS. 7A and 7B). Overall, these data establish that OSI-632 could inhibit multiple ‘key’ pro-fibrosis signaling proteins in vivo. Furthermore, these data also indicate that simultaneous inhibition of multiple parallel signaling pathways and/or multiple nodes in a single pathway can present benefits, such as improved efficacy and reduction of drug resistance.

Referring to FIGS. 7A and 7B, OSI-632 inhibits activation of multiple signaling pathways in vivo. FIG. 7A shows that OSI-632 treatment significantly reduced phosphorylation of indicated protein and total levels of collagen 1 in lung tissue collected from bleomycin induced IPF mouse model. Protein changes were measured using RPPA validated antibodies. FIG. 7B shows that OSI-632 inhibits bleomycin induced activation of STAT3. Top, Representative IHC images showing reduction in nuclear intensity of activated STAT3 (Y705) in response to OSI-632 treatment. Bottom, plots shown relative quantification of strong staining and H-score. Asterisk denotes statistical significance by one-way Anova compares to bleomycin treated group. * is p<0.05; ** p<0.01; *** p<0.001; **** p<0.0001. Bars represent means and error bars represent SEM. Thus, OSI-632 treatment inhibited multiple growth factor mediated signaling pathways important for myofibroblast activation, proliferation and function in lung tissue collected from bleomycin induced IPF mouse model.

Referring to FIG. 8, OSI-632 inhibited activation of multiple key pro-fibrotic factors in vivo. In particular, FIG. 8 shows changes in mRNA expression of indicated genes in fibrotic lung samples from bleomycin induced lung fibrosis mouse model. Asterisk denotes statistical significance by one-way Anova compares to bleomycin treated group. * is p<0.05; ** p<0.01; *** p<0.001; **** p<0.0001. Bars represent means and error bars represent SEM.

### Example 2. Non-Alcoholic Steatohepatitis Model

Liver fibrosis, a leading cause of morbidity and mortality worldwide, is characterized by the excessive accumulation of extracellular matrix (ECM) that obliterates the hepatic architecture and function by forming fibrous scars. It can be caused by chronic liver injury of virtually any etiology, including Nonalcoholic steatohepatitis (NASH). NASH is characterized by liver lobular inflammation and hepatocyte ballooning degeneration due to fat accumulation. It is estimated that 15-20 million people have NASH in the U.S., and the number is projected to be ˜30 million by 2030. Up to 60% of NASH patients have mild (F1 stage) and moderate (F2 stage) liver fibrosis. In addition, 20-30% of NASH cases are estimated to have severe fibrosis (F3) and cirrhosis (F4), including progression to HCC in some cases. Thus, NASH can severely affect life quality with devastating clinical outcomes, including organ transplant and mortality. The ultimate goal for any therapeutic intervention is to resolve fibrosis, resulting in a reduced need for organ transplants and decreased mortality. However, there are no drugs approved for treating NASH, representing a significant unmet need.

During liver fibrosis, the activation of hepatic stellate cells (HSCs), which are hepatic resident mesenchymal cells, contributes to the net production of ECM and induction of a pro-fibrotic microenvironment. Under physiological conditions, HSCs reside in the perisinusoidal space in between the hepatocytes and sinusoidal endothelial cells and maintain a quiescent state (qHSCs). qHSCs store retinoid lipid droplets and have extensive dendrite-like processes along the sinusoid. Following an injury in vivo or in vitro culturing, quiescent HSCs transdifferentiate into myofibroblasts. In this process, they lose their retinoid droplets, enlarge and flatten, express αSMA and vimentin and become more proliferative, migratory, and proinflammatory. They synthesize large quantities of extracellular matrix molecules (e.g., type I and III collagens) and other ECM remodeling proteins (e.g., MMPs and TIMPs). TGF-β is considered to be the most potent fibrogenic and proliferative signal to stimulate myofibroblast activation in the liver. The binding of TGF-β to its receptors and subsequent activation of its downstream component SMAD2/3 promotes fibrosis by prompting HSC differentiation into myofibroblasts, enhancing expression of tissue inhibitors of matrix metalloproteases (TIMPs) that block ECM degradation, and by directly promoting transcription of type I and type III collagens. Other signals, including growth factor signals, such as PDGF and VEGF have all been demonstrated in vitro and/or in vivo to dampen the activated phenotypes of HSCs and contribute to some degree of resolution of liver fibrosis and inflammation. The major clinical challenge posed by these myofibroblasts is progressive hepatic fibrosis, resulting in the requirement of a liver transplant, mortality, or the development of hepatocellular carcinoma.

OSI-632 Offers an Accelerated, Safer, and Effective Therapeutic Option for NASH

Without wishing to be bound by theory, it is believed that the biological rationale behind OSI-632 could potentially stop or reverse the fibrotic process and be a safer option for a broader patient population. First, OSI-632 comprehensively blocks signaling pathways critical to the activation and differentiation of fibroblasts and myofibroblasts. OSI-632 is an ATP-competitive small molecule that could inhibit VEGFR, that could potently inhibit FGFR and, to a lesser extent, inhibit PDGF signaling, all-important pathways to myofibroblast proliferation and function. More importantly, as will be described below, it was discovered that it strongly inhibits TGF-β signaling in fibroblasts and myofibroblasts and much more effectively than pirfenidone and galunisertib (known TGF-β signaling inhibitors), resulting in reduced motility, contractility as well as expression of αSMA, ECM molecules, and other pro-fibrotic molecules. Though the pathogenic mechanisms underlying NASH are not yet fully understood, a major culprit of NASH-induced fibrosis is TGF-β driven differentiation of HSCs into myofibroblasts and production of pro-fibrotic molecules, which promote ECM deposition.

Second, it is believed that OSI-632 will be safe and tolerable to NASH patients, especially when the liver abnormality is a concern. For example, in multiple phases I and II trials of OSI-632 for various cancers, OSI-632 was determined to be safe and well-tolerated with toxicities that are primarily low grade and readily managed with conventional medical measures at a maximal tolerable dose of 200 mg/day. See, Cohen, R. B. et al., A phase I/randomized phase II, non-comparative, multicenter, open label trial of CP-547,632 in combination with paclitaxel and carboplatin or paclitaxel and carboplatin alone as first-line treatment for advanced non-small cell lung cancer (NSCLC), 60, 81-89 (2007); and Tolcher, A. et al., 137 A phase I study of an oral vascular endothelial growth factor receptor-2 (VEGFR-2) tyrosine kinase inhibitor, CP-547,632, in patients with advanced solid tumors, 8, 44 (2004), the contents of each of which is herein incorporated in its entirety.

Notably, OSI-632 neither had toxicity issues of bleeding and thrombosis sometimes observed in studies of agents targeting VEGF/VEGFR, nor does it cause any adverse events in the liver. Overall, it is believed that OSI-632 can potentially reverse liver fibrosis while sparing severe toxicities to warrant its use for broader NASH patients.

OSI-632 as a Potent Inhibitor of Myofibroblast Function

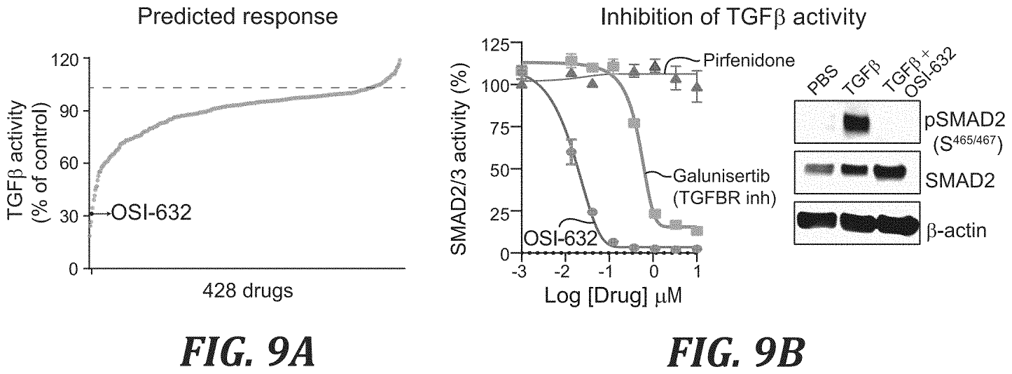

In preliminary studies, a well-established, culture-activated human hepatic stellate cell model (LX2, initially established by Scott Friedman as described in Xu, L. et al., Human hepatic stellate cell lines, LX-1 and LX-2: new tools for analysis of hepatic fibrosis. Gut 54, 142-151 (2005), incorporated herein by reference in its entirety) was used and a set of 32 computationally chosen, broad-specific kinase inhibitors that collectively target a broad range of kinases were screened. Each drug was examined at eight concentrations, and its effect on the activity of TGF-β (measured by SMAD2/3 luciferase reporter) and on aHSC cell viability was scored. 1536 data points were then analyzed to develop regression modeling to predict the response to >400 drugs (FIG. 9A). 11 drugs that could inhibit TGF-β activity in LX2 cells by >50% of DMSO control were identified. Of these, the efficacy of OSI-632 to block TGF-β activity in LX2 cells (IC₅₀ 13.7 nM) was validated (FIG. 9B). Importantly, treatment with OSI-632 outperformed galunisertib (a known TGFBR inhibitor) and pirfenidone, an FDA-approved TGF-β antagonist for pulmonary fibrosis. (FIG. 9B). Further, kinome-profiling of OSI-632 revealed that it is a potent inhibitor of FGFR (90% inhibition at 500 nM), VEGFR (˜75% inhibition at 500 nM), and TGFBR1/2 (91% inhibition at 500 nM). Overall, the screening effort identified and validated that OSI-632, a clinical-grade compound, inhibited TGF-β activity in activated hepatic stellate cells.

OSI-632 Blocks HSC Motility and Expression of Marker Genes of Myofibroblast Activation

Methods:

LX2 cells were obtained from Sigma Aldrich (Cat #SCC064). LX2 cells were grown at 37° C. under 5% CO2, 95% ambient atmosphere and maintained in Dulbecco's minimum essential medium (DMEM) supplemented with 10% FBS (Sigma) and 1% Penn Strep.

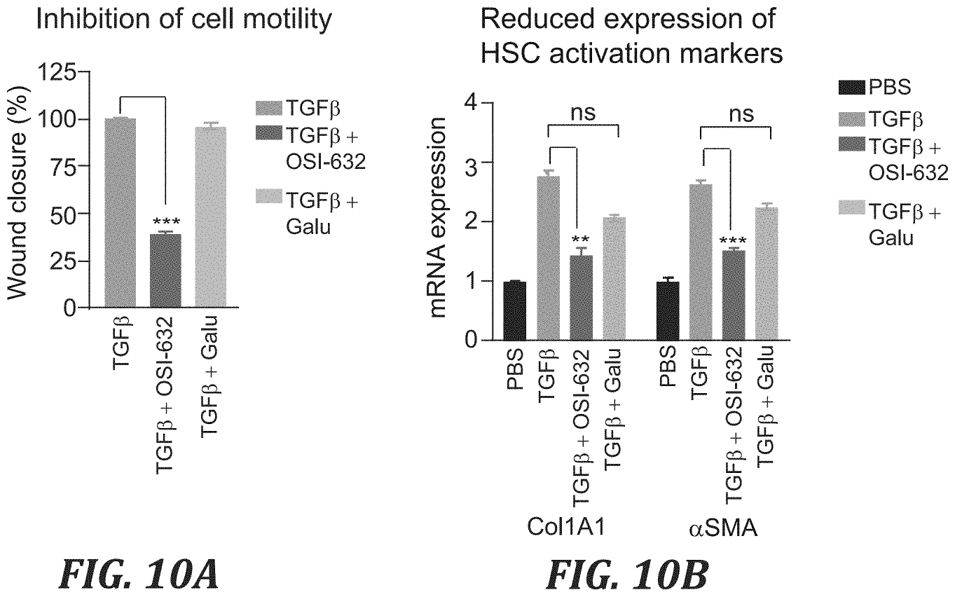

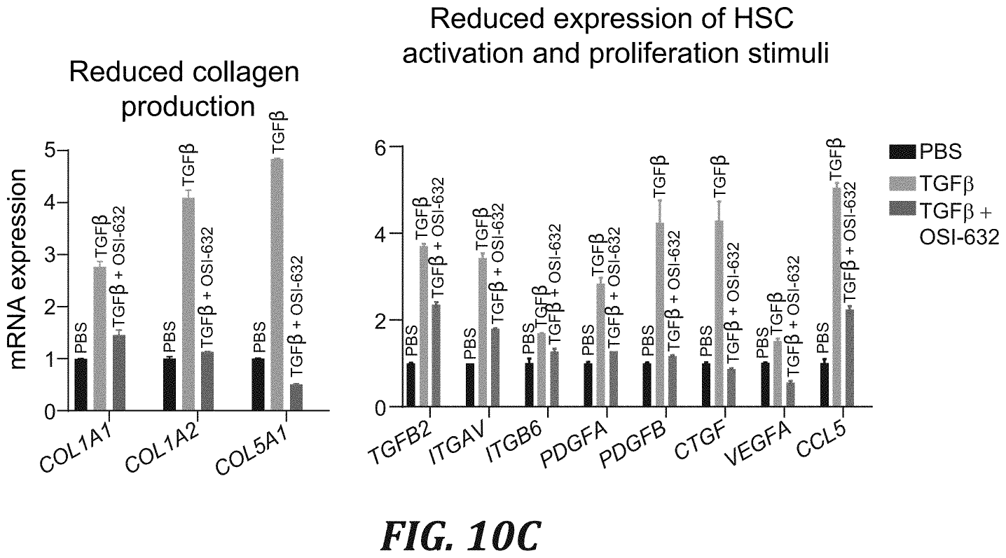

FIG. 10A: LX2 cells were plated on 96-well plates (Essen Image Lock, Essen Instruments). 24 hours later, TGFb at 6.5 ng/ml alone or TGFb with OSI-632 or galunisertib at various concentrations was added and a wound was scratched with wound scratcher (Essen Instruments). Wound closure was monitored with Incucyte Live-Cell Imaging System and software (Essen Instruments) every 2-4 hours for 72 hours. The mean relative wound density of at least three biological replicates was calculated. The wound density correlates with the cell motility. Plotted is the wound closure density at 50 hour.

FIGS. 10B and 10C: LX2 cells were incubated with TGF-β alone at 6.5 ng/ml or TGF-β with indicated drugs for 48 hours. Total cellular RNA was isolated using a RNeasy Mini Kit (QIAGEN). 1 μg of total RNA was reverse transcribed into first-strand cDNA using an RT2 First Strand Kit (QIAGEN). The resultant cDNA was subjected to qPCR using indicated human primers to measure the changes in mRNA expression of indicated human genes.

Activation of HSC is characterized by an increase in cell motility, contractility, and mitotic index. aHSCs upregulates expression of various integrins and ECM proteins that regulate cell adhesion, migration, and proliferation. In addition, integrins such as αV can control the release and activation of TGF-β and further promote fibrogenic phenotype.

Whether OSI-632 treatment inhibited TGF-β-mediated cell motility and expression of ECM protein and proliferation stimuli in human LX2 cells were investigated. The data show that 1 μM OSI-632 treatment OSI-632 treatment significantly decreased TGF-β-induced motility of LX2 cells (FIG. 10A). In contrast, treatment with galunisertib at therapeutically equivalent doses (a known TGFBR inhibitor) did not affect LX2 cell motility at the same dose. In addition, a significant decrease in TGF-β-induced expression of HSC activation markers such as COL1A1 (2-fold) and αSMA (2-fold) in LX2 cells was observed (FIG. 10B). Again, treatment with galunisertib had a modest effect on both COL1A1 and αSMA. Further, the expression of several collagen proteins critical for forming collagen fibers was significantly inhibited in OSI-632 treated LX2 cells. These include COL1A1 (2-fold), COL1A2 (3-fold), and COL5A1 (5-fold) (FIG. 10C). Finally, a significant decrease in the expression of integrins (ITGAV and ITGB6) and HSC proliferation stimuli such as TGFB2, PDGFA, PDGFB, CTGF, VEGFA, and CCL5 was observed (FIG. 10C). Together, these compelling data demonstrated that OSI-632 comprehensively inhibited HSC activated phenotype.

OSI-632 Significantly Reduced Collagen Deposition and Stimulated Hepatocyte Regeneration In Vivo

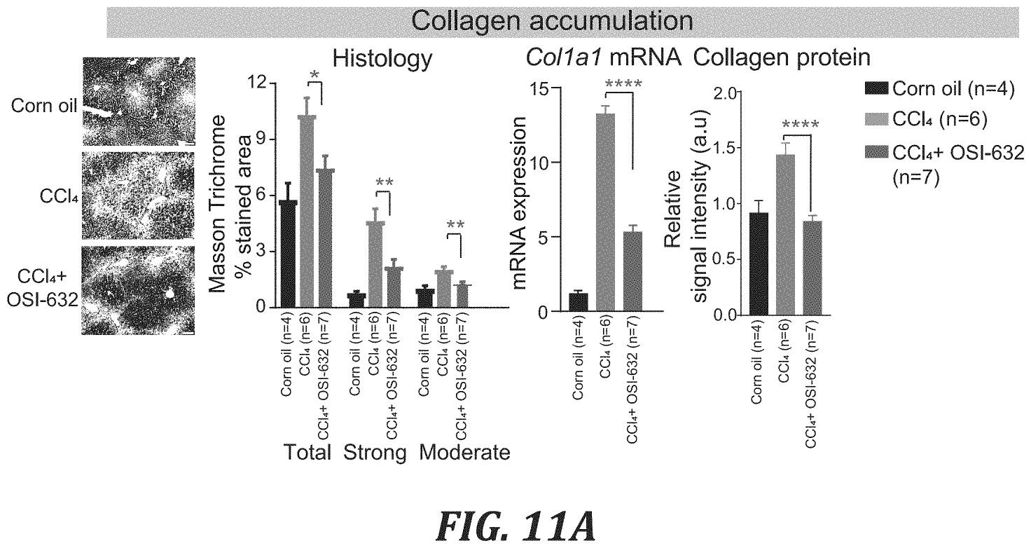

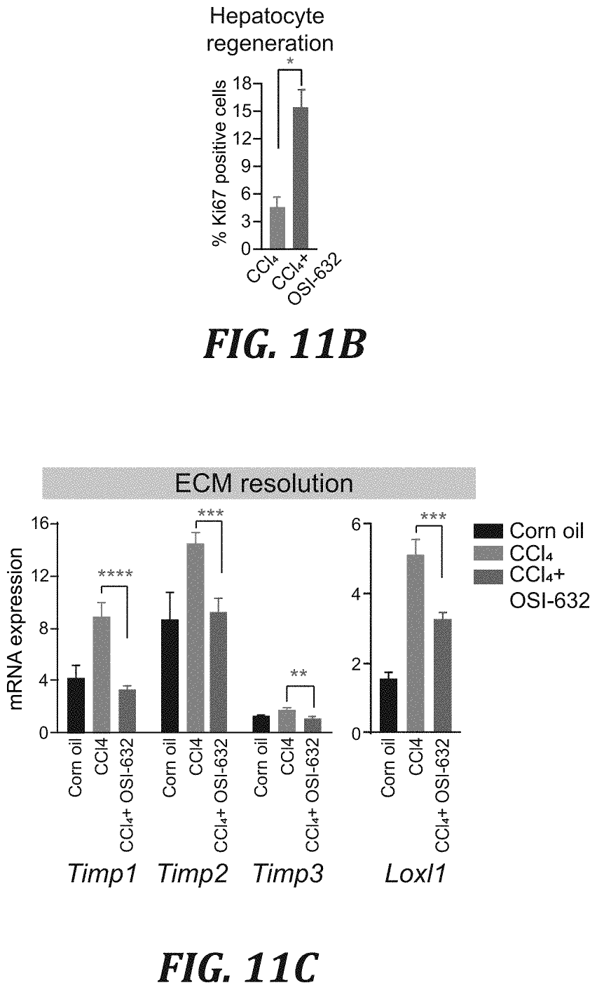

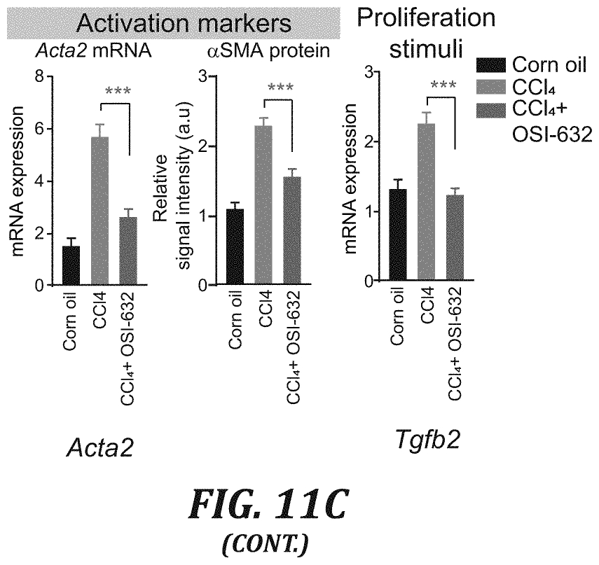

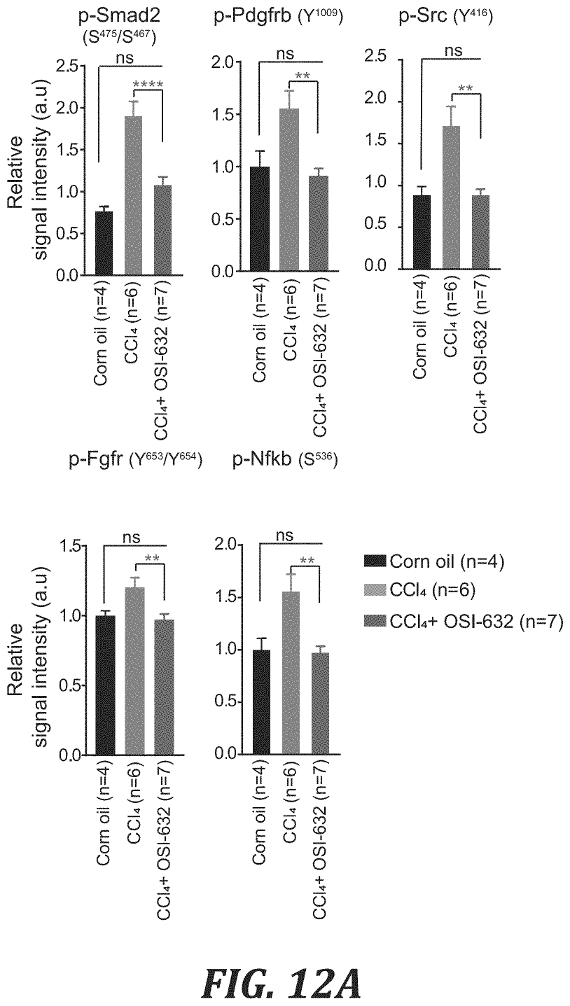

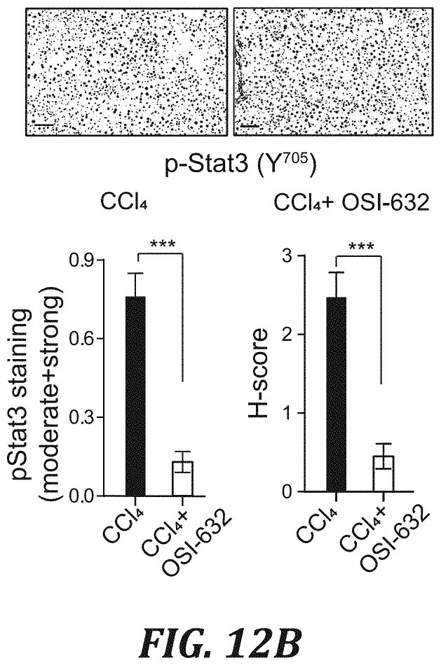

FIGS. 11A and 11B. Male C57BL/6J mice between 6-8 week old were used in the study. Mice were treated with intraperitoneal injection of 0.5 μl carbon tetrachloride (CCl₄) per gram body weight, dissolved in corn oil at a ratio of 1:3, twice a week (Tuesdays and Saturdays) for 5.5 weeks or corn oil control only. After 2.5 week, mice were treated with OSI-632 (80 mg/Kg) thrice a week for 3 weeks (Mondays, Wednesdays, and Fridays) or vehicle control via oral gavage. At the end of the study, livers were either snap frozen or fixed in 10% formalin and stained for H&E, Masson Trichrome (FIG. 11A) and Ki67 (FIG. 11B).

Total RNA was extracted from the snap frozen liver tissue in TRIzol™ Reagent by Omni Bead Ruptor homogenizer. 1 μg of total RNA was reverse transcribed into first-strand cDNA using an RT2 First Strand Kit (QIAGEN). The resultant cDNA was subjected to qPCR using indicated mouse primers to measure the changes in mRNA expression of indicated mouse genes (FIG. 11A and FIG. 11C).

Next, whether OSI-632 could inhibit liver fibrosis in mice was evaluated. In preliminary data, treatment with OSI-632 (80 mg/Kg) thrice/week (Mondays, Wednesdays, Fridays) caused a significant decrease in carbon tetrachloride (CCl₄)-induced fibrosis (FIGS. 11A-11C). Masson trichome staining showed a significant reduction in collagen deposition in OSI-632 treated group compared with CCl₄ only treated mice (FIG. 11A). Consistently, both mRNA expression of Col1a1 (measured by qPCR) and protein level of collagen 1 (measured by reverse-phase protein array; RPPA) was significantly decreased in OSI-632 treated mice compared with CCl₄-only treated controls (FIG. 11A). Importantly, increased hepatocyte regeneration was also observed as measured by Ki67 staining in livers treated with OSI-632 (FIG. 11B). Further, treatment with OSI-632 could also reverse CCl₄-induced expression of genes involved in ECM remodeling, Timp1 (5-fold), Timp2 (2-fold), Timp3 (˜2-fold), and Loxl1 (2-fold), HSC activation marker, Acta2 and proliferation stimuli such as Tgfb2 (FIG. 10C). Together, these data indicate that OSI-632 inhibited ECM remodeling, collagen deposition, expression of HSC activation and proliferation stimuli, and promoted hepatocyte regeneration in the CCl₄-induced fibrosis model.

OSI-632 Targets Multiple Signaling Pathways in the Fibrotic Liver

FIG. 12A: Total protein was extracted from the snap frozen liver tissue in T-PER™ Tissue Protein Extraction Reagent by Omni Bead Ruptor homogenizer. Changes in the phosphorylation levels of indicated proteins were detected in the liver homogenates from the CCl₄ in vivo studies by reverse-phase protein array.

FIG. 12B: pSTAT3 levels were detected in the fixed lung tissue by immunohistochemistry. Intensity of staining was quantified by HALO imaging software.

Next, OSI-632 target engagement in vivo was evaluated. Molecular analyses of CCl₄-treated mouse livers showed increased levels of phosphorylation of Smad2 (TGFb downstream effector), pro-fibrosis growth factor receptors including Pdgfrb and Fgfr, and their downstream effectors, including phosphorylated Src, Nfκβ, and STAT3 compared with corn oil-treated control cohort (FIGS. 12A and 12B). Consistent with in vitro HSC activation data (FIGS. 11A-11C), OSI-632 treatment significantly decreased CCl₄-induced phosphorylation of Smad2, Pdgfrb, Fgfr, Src, Nfκβ, and STAT3 compared with CCl₄-only treated mouse livers tissues (FIGS. 12A and 12B). Overall, these data establish that OSI-632 could inhibit multiple ‘key’ pro-fibrosis signaling proteins in vivo. Furthermore, these data also indicate that simultaneous inhibition of multiple parallel signaling pathways and/or multiple nodes in a single pathway can have benefits, such as improved efficacy and reduction of drug resistance.

By example and without limitation, embodiments are disclosed according to the following enumerated paragraphs:
A1. A method of inhibiting signaling mediated by TGF-β, FGF, VEGF, PDGF, and/or a Src family kinase in a cell, comprising contacting the cell with a therapeutically effective amount of a compound of formula (I)

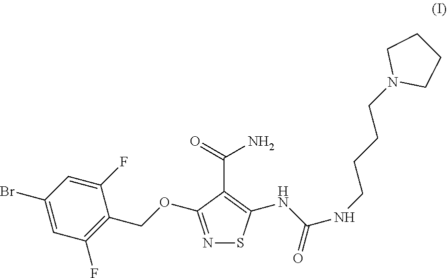 [SMILES: NC(=O)c1c(OCc2c(F)cc(Br)cc2F)nsc1NC(=O)NCCCCN1CCCC1]

or a pharmaceutically acceptable salt, prodrug, or solvate thereof.
A2. The method of paragraph A1, wherein the cell is a myofibroblast or fibroblast transdifferentiated from any cell type.
A3. The method of paragraph A2, wherein the myofibroblast or fibroblast comprises a liver myofibroblast, a lung myofibroblast, a kidney myofibroblast, or a cardiac myofibroblast.
A4. The method of paragraph A3, wherein the liver myofibroblast is transdifferentiated from a hepatic stellate cell (HSC).
A5. The method of any one of paragraphs A1 to A4, wherein the compound, or a pharmaceutically acceptable salt, prodrug, or solvate thereof, decreases the expression of TGF-01, CTGF, IL-11, FGF, VEGF, FN1, or any combination thereof.
A6. The method of any one of paragraphs A1 to A5, wherein the compound, or a pharmaceutically acceptable salt, prodrug, or solvate thereof, inhibits activation of SMAD2/3, Erk, and STAT3 mediated by TGF-β in the cell.
A7. The method of any one of paragraphs A1 to A6, wherein the compound, or a pharmaceutically acceptable salt, prodrug, or solvate thereof, inhibits expression of a downstream signaling protein mediated by TGF-β, FGF, VEGF, PDGF, and/or a Src family kinase.
A8. The method of any one of paragraphs A1 to A7, wherein the compound, or a pharmaceutically acceptable salt, prodrug, or solvate thereof, inhibits expression of αSMA, COL1A1, COL1A2, COL3A1, COL5A1, PDGFA, PDGFB, CTGF, IL11, VEGFA, CCL5, FN1, or any combination thereof.
A9. The method of any one of paragraphs A1 to A8, wherein the compound, or a pharmaceutically acceptable salt, prodrug, or solvate thereof, inhibits phosphorylation of Src, Erk, JNK, S6, Nfκβ, STAT3, or any combination thereof.
A10. The method of any one of paragraphs A1 to A9, wherein the compound, or a pharmaceutically acceptable salt, prodrug, or solvate thereof, inhibits the expression of Timp1, Timp2, Timp3, Loxl1, or any combination thereof.
A11. The method of one of paragraphs A1 to A10, wherein the cell is in an in vitro culture and contacting the cell comprises adding the compound, or a pharmaceutically acceptable salt, prodrug, or solvate thereof, to the in vitro culture.
A12. The method of one of paragraphs A1 to A10, wherein the cell is in vivo in a subject with a condition treatable by inhibiting signaling mediated by TGF-β, FGF, VEGF, PDGF, and/or a Src family kinase, wherein the method comprises administering to the subject a therapeutically effective amount of a pharmaceutical composition comprising:
the compound of Formula (I), or a pharmaceutically acceptable salt, prodrug, or solvate thereof, and
a pharmaceutically acceptable carrier.
A13. The method of any one of paragraphs A1 to A12, wherein administering comprises oral administration.

A14. The method of any one of paragraphs A1 to A14, comprising administering the compound, or a pharmaceutically acceptable salt, prodrug, or solvate thereof, at a dosage equivalent to from 10 mg compound/kg to 100 mg compound/kg.

A15. A method of inhibiting activity of a myofibroblast or fibroblast, comprising contacting the myofibroblast or fibroblast with a therapeutically effective amount of a compound of formula (I)

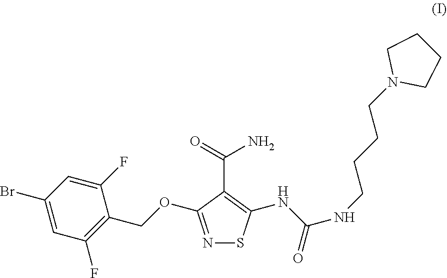 [SMILES: NC(=O)c1c(OCc2c(F)cc(Br)cc2F)nsc1NC(=O)NCCCCN1CCCC1]

or a pharmaceutically acceptable salt, prodrug, or solvate thereof.
A16. The method of paragraph A15, wherein the myofibroblast or fibroblast in a liver, a lung myofibroblast or fibroblast, a kidney myofibroblast, a cardiac myofibroblast, or a combination thereof.
A17. The method of paragraph A16, wherein the liver myofibroblast is transdifferentiated from a hepatic stellate cell (HSC).
A18. The method of paragraph A16 or paragraph A17, wherein inhibiting activity comprises inhibiting contractility and/or motility of the myofibroblast or fibroblast.
A19. The method of any one of paragraphs A15 to A18, wherein inhibiting activity comprises inhibiting deposition of extracellular matrix (ECM) by the myofibroblast or fibroblast.
A20. The method of any one of paragraphs A15 to A19, wherein the cell is in an in vitro culture and the method comprises adding the compound, or a pharmaceutically acceptable salt, prodrug, or solvate thereof, to the culture.
A21. The method of any one of paragraphs A15 to A19, wherein the cell is in vivo in a subject with a condition treatable by inhibiting activation of a myofibroblast or fibroblast, wherein the method comprises administering to the subject a therapeutically effective amount of a pharmaceutical composition comprising:
the compound of Formula (I), or a pharmaceutically acceptable salt, prodrug, or solvate thereof, and
a pharmaceutically acceptable carrier.
A22. The method of paragraph A21, wherein the subject has a fibrosis condition.
A23. The method of paragraph A22, wherein the fibrosis condition is, or is associated with, non-alcoholic steatohepatitis (NASH), cirrhosis, HBV infection, any liver disease, pulmonary fibrosis, interstitial lung disease, idiopathic pulmonary fibrosis (IPF), renal fibrosis, cardiac fibrosis, or any combination thereof.
A24. The method of paragraph A22, wherein the fibrosis condition comprises a liver disease.
A25. A method of increasing hepatocyte regeneration in a subject in need thereof, comprising administering to the subject a therapeutically effective amount of a pharmaceutical composition comprising a compound of Formula (I)

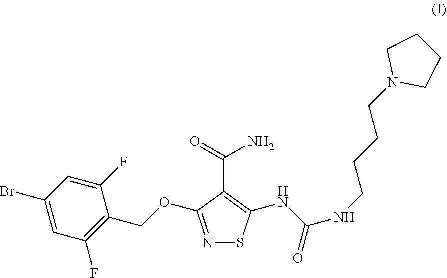 [SMILES: NC(=O)c1c(OCc2c(F)cc(Br)cc2F)nsc1NC(=O)NCCCCN1CCCC1]

or a pharmaceutically acceptable salt, prodrug, or solvate thereof, and a pharmaceutically acceptable carrier.
A26. The method of paragraph A25, wherein the subject has a fibrosis condition.
A27. The method of paragraph A26, wherein the fibrosis condition is, or is associated with, non-alcoholic steatohepatitis (NASH), cirrhosis, HBV infection, or any liver disease.
A28. The method of any one of paragraphs A25 to A27, wherein the compound, or a pharmaceutically acceptable salt, prodrug, or solvate thereof, inhibits collagen deposition, ECM remodeling, promotes hepatocyte regeneration or a combination thereof, in a fibrotic liver tissue of the subject.
A29. The method of any one of paragraphs A25 to A28, wherein the compound, or a pharmaceutically acceptable salt, prodrug, or solvate thereof, decreases phosphorylation of Smad2, Pdgfrb, Fgfr, Src, Nfκβ, STAT3, or any combination thereof, in a fibrotic liver, fibrotic lung tissue, liver myofibroblast, lung myofibroblast, liver fibroblast, lung fibroblast, or any combination thereof, of the subject.
A30. A method of treating a fibrosis condition in a subject in need thereof, comprising administering to subject a therapeutically effective amount of a first pharmaceutical composition comprising:
a compound of formula (I)

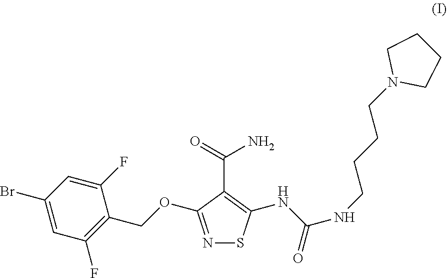 [SMILES: NC(=O)c1c(OCc2c(F)cc(Br)cc2F)nsc1NC(=O)NCCCCN1CCCC1]

or a pharmaceutically acceptable salt, prodrug, or solvate thereof, and
a pharmaceutically acceptable carrier.
A31. The method of paragraph A30, wherein the fibrosis condition is, or is associated with, non-alcoholic steatohepatitis (NASH), cirrhosis, HBV infection, any liver disease, pulmonary fibrosis/interstitial lung disease, idiopathic pulmonary fibrosis (IPF), renal fibrosis, or cardiac fibrosis.
A32. The method of paragraph A31, wherein the fibrosis condition is idiopathic pulmonary fibrosis or pulmonary fibrosis/interstitial lung disease and the compound, or a pharmaceutically acceptable salt, prodrug, or solvate thereof, inhibits collagen deposition, interstitial septal thickening, collapse of air spaces, inflammation, or any combination thereof, in a pulmonary fibrotic tissue.
A33. The method of any one of paragraphs A30 to A32, wherein administering comprises oral administration.
A34. The method of any one of paragraphs A30 to A33, comprising administering the compound, or a pharmaceutically acceptable salt, prodrug, or solvate thereof, at a dosage equivalent to from 10 mg compound/kg to 100 mg compound/kg.
A35. The method of any one of paragraphs A30 to A34, further comprising administering the subject with a therapeutically effective amount of a second pharmaceutical composition to treat fibrosis.
A36. The method of paragraph A35, comprising administering the first pharmaceutical composition before, simultaneously with, or after administering the second pharmaceutical composition.
A37. The method of paragraph A35 or paragraph A36, wherein the second composition comprises nintedanib, pirfenidone, GLPF1690, pamreclumab, obeticholic acid (OCA), elafabranor, cenicriviroc, or any combination thereof.
A38. The method of any one of paragraphs A30 to A37, wherein the first pharmaceutical composition is administered multiple times over a period of a day, a week, or a month.

While illustrative embodiments have been illustrated and described, it will be appreciated that various changes can be made therein without departing from the spirit and scope of the invention.

1. A method of inhibiting signaling mediated by TGF-β, FGF, VEGF, PDGF, and/or a Src family kinase in a cell, comprising contacting the cell with a therapeutically effective amount of a compound of formula (I) 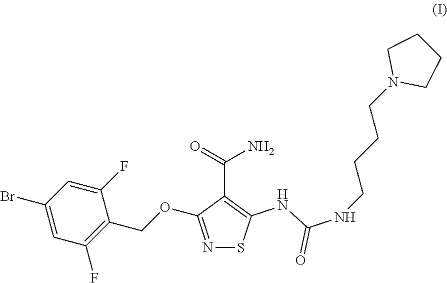 [SMILES: NC(=O)c1c(OCc2c(F)cc(Br)cc2F)nsc1NC(=O)NCCCCN1CCCC1]
or a pharmaceutically acceptable salt, prodrug, or solvate thereof.

1. The method of claim 1, wherein the cell is a myofibroblast or fibroblast transdifferentiated from any cell type.

2. The method of claim 2, wherein the myofibroblast or fibroblast comprises a liver myofibroblast, a lung myofibroblast, a kidney myofibroblast, or a cardiac myofibroblast.

3. The method of claim 3, wherein the liver myofibroblast is transdifferentiated from a hepatic stellate cell (HSC).

4. The method of claim 1, wherein the compound, or a pharmaceutically acceptable salt, prodrug, or solvate thereof, decreases the expression of TGF-β1, CTGF, IL-11, FGF, VEGF, FN1, or any combination thereof.

5. The method of claim 1, wherein the compound, or a pharmaceutically acceptable salt, prodrug, or solvate thereof, inhibits activation of SMAD2/3, Erk, and STAT3 mediated by TGF-β in the cell.

6. The method of claim 1, wherein the compound, or a pharmaceutically acceptable salt, prodrug, or solvate thereof, inhibits expression of a downstream signaling protein mediated by TGF-β, FGF, VEGF, PDGF, and/or a Src family kinase.

7. The method of claim 1, wherein the compound, or a pharmaceutically acceptable salt, prodrug, or solvate thereof, inhibits expression of αSMA, COL1A1, COL1A2, COL3A1, COL5A1, PDGFA, PDGFB, CTGF, IL11, VEGFA, CCL5, FN1, or any combination thereof.

8. The method of claim 1, wherein the compound, or a pharmaceutically acceptable salt, prodrug, or solvate thereof, inhibits phosphorylation of Src, Erk, JNK, S6, Nfκβ, STAT3, or any combination thereof.

9. The method of claim 1, wherein the compound, or a pharmaceutically acceptable salt, prodrug, or solvate thereof, inhibits the expression of Timp1, Timp2, Timp3, Loxl1, or any combination thereof.

10. The method of claim 1, wherein the cell is in an in vitro culture and contacting the cell comprises adding the compound, or a pharmaceutically acceptable salt, prodrug, or solvate thereof, to the in vitro culture.

11. The method of claim 1, wherein the cell is in vivo in a subject with a condition treatable by inhibiting signaling mediated by TGF-β, FGF, VEGF, PDGF, and/or a Src family kinase, wherein the method comprises administering to the subject a therapeutically effective amount of a pharmaceutical composition comprising:
the compound of Formula (I), or a pharmaceutically acceptable salt, prodrug, or solvate thereof, and
a pharmaceutically acceptable carrier.

12. The method of claim 1, wherein administering comprises oral administration.

13. The method of claim 1, comprising administering the compound, or a pharmaceutically acceptable salt, prodrug, or solvate thereof, at a dosage equivalent to from 10 mg compound/kg to 100 mg compound/kg.

14. A method of inhibiting activity of a myofibroblast or fibroblast, comprising contacting the myofibroblast or fibroblast with a therapeutically effective amount of a compound of formula (I) 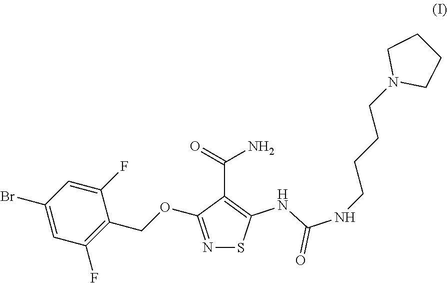 [SMILES: NC(=O)c1c(OCc2c(F)cc(Br)cc2F)nsc1NC(=O)NCCCCN1CCCC1]
or a pharmaceutically acceptable salt, prodrug, or solvate thereof.

15. The method of claim 15, wherein the myofibroblast or fibroblast in a liver, a lung myofibroblast or fibroblast, a kidney myofibroblast, a cardiac myofibroblast, or a combination thereof.

16. The method of claim 16, wherein the liver myofibroblast is transdifferentiated from a hepatic stellate cell (HSC).

17. The method of claim 16, wherein inhibiting activity comprises inhibiting contractility and/or motility of the myofibroblast or fibroblast.

18. The method of claim 15, wherein inhibiting activity comprises inhibiting deposition of extracellular matrix (ECM) by the myofibroblast or fibroblast.

19. The method of claim 15, wherein the cell is in an in vitro culture and the method comprises adding the compound, or a pharmaceutically acceptable salt, prodrug, or solvate thereof, to the culture.

20. The method of claim 15, wherein the cell is in vivo in a subject with a condition treatable by inhibiting activation of a myofibroblast or fibroblast, wherein the method comprises administering to the subject a therapeutically effective amount of a pharmaceutical composition comprising:
the compound of Formula (I), or a pharmaceutically acceptable salt, prodrug, or solvate thereof, and
a pharmaceutically acceptable carrier.

21. The method of claim 21, wherein the subject has a fibrosis condition.

22. The method of claim 22, wherein the fibrosis condition is, or is associated with, non-alcoholic steatohepatitis (NASH), cirrhosis, HBV infection, any liver disease, pulmonary fibrosis, interstitial lung disease, idiopathic pulmonary fibrosis (IPF), renal fibrosis, cardiac fibrosis, or any combination thereof.

23. The method of claim 22, wherein the fibrosis condition comprises a liver disease.

24. A method of increasing hepatocyte regeneration in a subject in need thereof, comprising administering to the subject a therapeutically effective amount of a pharmaceutical composition comprising a compound of Formula (I) 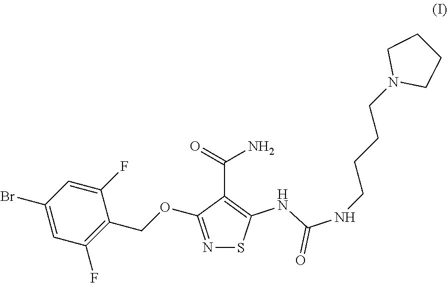 [SMILES: NC(=O)c1c(OCc2c(F)cc(Br)cc2F)nsc1NC(=O)NCCCCN1CCCC1]
or a pharmaceutically acceptable salt, prodrug, or solvate thereof, and a pharmaceutically acceptable carrier.

25. The method of claim 25, wherein the subject has a fibrosis condition.

26. The method of claim 26, wherein the fibrosis condition is, or is associated with, non-alcoholic steatohepatitis (NASH), cirrhosis, HBV infection, or any liver disease.

27. The method of claim 25, wherein the compound, or a pharmaceutically acceptable salt, prodrug, or solvate thereof, inhibits collagen deposition, ECM remodeling, promotes hepatocyte regeneration or a combination thereof, in a fibrotic liver tissue of the subject.

28. The method of claim 25, wherein the compound, or a pharmaceutically acceptable salt, prodrug, or solvate thereof, decreases phosphorylation of Smad2, Pdgfrb, Fgfr, Src, Nfκβ, STAT3, or any combination thereof, in a fibrotic liver, fibrotic lung tissue, liver myofibroblast, lung myofibroblast, liver fibroblast, lung fibroblast, or any combination thereof, of the subject.

29. A method of treating a fibrosis condition in a subject in need thereof, comprising administering to subject a therapeutically effective amount of a first pharmaceutical composition comprising:
a compound of formula (I) 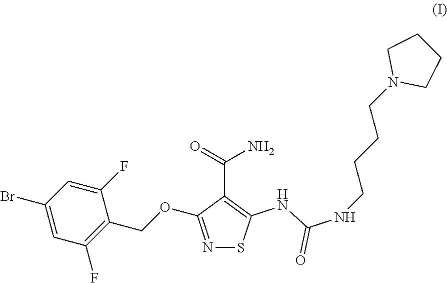 [SMILES: NC(=O)c1c(OCc2c(F)cc(Br)cc2F)nsc1NC(=O)NCCCCN1CCCC1]
or a pharmaceutically acceptable salt, prodrug, or solvate thereof, and
a pharmaceutically acceptable carrier.

30. The method of claim 30, wherein the fibrosis condition is, or is associated with, non-alcoholic steatohepatitis (NASH), cirrhosis, HBV infection, any liver disease, pulmonary fibrosis/interstitial lung disease, idiopathic pulmonary fibrosis (IPF), renal fibrosis, or cardiac fibrosis.

31. The method of claim 31, wherein the fibrosis condition is idiopathic pulmonary fibrosis or pulmonary fibrosis/interstitial lung disease and the compound, or a pharmaceutically acceptable salt, prodrug, or solvate thereof, inhibits collagen deposition, interstitial septal thickening, collapse of air spaces, inflammation, or any combination thereof, in a pulmonary fibrotic tissue.

32. The method of claim 30, wherein administering comprises oral administration.

33. The method of claim 30, comprising administering the compound, or a pharmaceutically acceptable salt, prodrug, or solvate thereof, at a dosage equivalent to from 10 mg compound/kg to 100 mg compound/kg.

34. The method of claim 30, further comprising administering the subject with a therapeutically effective amount of a second pharmaceutical composition to treat fibrosis.

35. The method of claim 35, comprising administering the first pharmaceutical composition before, simultaneously with, or after administering the second pharmaceutical composition.

36. The method of claim 35, wherein the second composition comprises nintedanib, pirfenidone, GLPF1690, pamreclumab, obeticholic acid (OCA), elafabranor, cenicriviroc, or any combination thereof.
